(19) United States
(12) Patent Application Publication
(10) Pub. No.: US 2025/0041553 A1
(43) Pub. Date: Feb. 6, 2025

(54) BRAIN STIMULATION SYSTEMS AND METHODS

(71) Applicant: GEORGIA TECH RESEARCH CORPORATION, Atlanta, GA (US)

(72) Inventors: Annabelle C. SINGER, Atlanta, GA (US); Tina C. FRANKLIN, Atlanta, GA (US)

(21) Appl. No.: 18/723,641

(22) PCT Filed: Dec. 22, 2022
(86) PCT No.: PCT/US2022/053825

§ 371 Date: Jun. 24, 2024

Related U.S. Application Data
(60) Provisional application No. 63/293,344, filed on Dec. 23, 2021.

(51) Int. Cl. A61M 21/00

(52) U.S. Cl. CPC A61M 21/00; A61M 2021/0027; A61M 2021/0044

(57) ABSTRACT

A method for treating a neuropsychiatric or neurological disorder by inducing neurological response in a subject, the method comprising: selecting a frequency for application of non-invasive brain stimulation to which a subject is to be exposed; exposing the subject to the non-invasive sensory stimulation at the selected frequency in a range of about 5-100 Hz.

### RELATED APPLICATION

This PCT application claims priority to, and the benefit of, U.S. Provisional Patent Application No. 63/293,344 filed Dec. 23, 2021, entitled “BRAIN STIMULATION SYSTEMS AND METHODS”, which is incorporated by reference herein in its entirety.

### TECHNICAL FIELD

The present disclosure pertains to systems and methods for using brain stimulation.

### BACKGROUND

Individuals that have suffered from chronic or severe stress have a two-fold or greater increased risk of developing neurodegenerative disease and are more likely to exhibit neuropsychiatric symptoms like anxiety and anhedonia. Psychosocial stress is thought to increase risk of dementia and neuropsychiatric symptoms and disease in part through synaptic loss and immune deregulation. Halting stress effects in the prodromal or early stages of disease could significantly reduce risk for the onset and progression of dementia, neuropsychiatric disease, and other stress-induced pathologies by modulating glia mediated changes in synaptic integrity. Furthermore, halting the detrimental effects of stress increases well-being and functional performance regardless of disease status.

While pharmacological, exercise, and other lifestyle interventions improve brain function and protect against some of the pathophysiological effects of chronic stress, low adherence to exercise regimen and undesirable pharmacological side effects have rendered these interventions ineffective in practice. Therefore, an urgent unmet need exists for therapies that increase resilience to stress and prevent stress pathophysiology to reduce the incidence of stress-induced pathologies and neuropsychiatric symptoms in such pathologies.

### SUMMARY

Provided herein are methods for increasing resilience to stress-induced pathology in a subject comprising exposing the subject to brain stimulation that is effective to drive oscillations, rhythmic electrical activity, or both in the subject at a frequency of about 5-100 Hz, wherein the subject is not currently subject to a diagnosis of a neurological or neuropsychiatric disorder, and, wherein the subject is selected for exposure to the brain stimulation based on (i) the subject's previous exposure to a stressor, (ii) the subject's contemporaneous exposure to a stressor, or (iii) an anticipated exposure of the subject to a future stressor.

Also disclosed are methods for increasing resilience to stress-induced pathology in a subject comprising exposing the subject to brain stimulation that is effective to drive oscillations, rhythmic electrical activity, or both in the subject at a frequency of about 5-100 Hz, wherein the subject is not currently subject to a diagnosis of a neurological or neuropsychiatric disorder, and wherein the subject has been assessed as having an elevated genetic risk for a neurological or neuropsychiatric disorder.

The present disclosure also provides methods for treating a neurological or neuropsychiatric disorder in a subject for whom an antidepressant or anti-anxiety medication is contraindicated or who is resistant to treatment with an antidepressant or anti-anxiety medication comprising exposing the subject to brain stimulation that is effective to drive oscillations, rhythmic electrical activity, or both in the subject at a frequency of about 5-100 Hz.

Also disclosed herein are methods for reducing dysregulation of classic complement signaling, dysregulation of microglia activity, neuroinflammation, or pathological synaptic change in a subject comprising exposing the subject to brain stimulation that is effective to drive oscillations, rhythmic electrical activity, or both, in the subject at a frequency of about 5-100 Hz, wherein the subject is selected for exposure to the brain stimulation based on a determination of neuroinflammation, dysregulation of classic complement signaling, dysregulation of microglia activity, or synaptic loss in the subject, or, wherein the subject is selected for exposure to the brain stimulation based on a determination that the subject has an elevated genetic risk for a condition having a classic complement component.

### BRIEF DESCRIPTION OF THE DRAWINGS

FIG. 1 depicts how sensory stimulation, including sensory flicker, promotes resilience to stress.

FIG. 2A provides the results of a study demonstrating that audio-visual flicker promotes stress-resilient behavior.

FIG. 2B illustrates how assessment of stress-related behaviors is conducted.

FIG. 3 illustrates how audiovisual flicker upregulates synaptic genes.

FIG. 4A addresses whether chronic stress alters microglia.

FIG. 4B addresses whether different frequencies of brain stimulation alters microglia.

FIG. 5A illustrates how brain stimulation reduces expression of microglia phagocytosis gene CD68 in a mouse model.

FIG. 5B illustrates how brain stimulation suppresses classic complement signaling gene C3 and upregulated serpinG1, which inhibits classic complement signaling in a mouse model.

FIG. 6A provides the results of a study demonstrating that the effects of stress on anxiety, measured via elevate plus maze, require microglia in males.

FIG. 6B shows how microglia were successfully depleted with PLX3397.

FIG. 7 illustrates changes in synaptogenesis signaling pathways following stress and flicker intervention. Region specific changes in synaptogenesis signaling pathway are reversed by audio-visual flicker in male and female mice.

FIG. 8A shows a study concerning the ability of brain stimulation to entrain human neurological circuits for an individual participant

FIG. 8B shows brain stimulation entrains human neurological circuits across multiple frequencies and individuals.

FIG. 9 illustrates audio-visual flicker induction of up-regulation of synaptic genes.

FIG. 10 illustrates preliminary data showing that sensory flicker stimulation leads to a reduction in stress-induced neuropsychiatric-like behavior.

FIG. 11 illustrates a model to show sensory flicker methods boost resilience to stress-induced pathology.

FIG. 12 illustrates how audiovisual flicker prevents stress induced astrocyte reactivity.

FIG. 13 illustrates capturing glia-neuron interaction with Super Resolution Confocal Microscopy STED.

FIG. 14 illustrates a methods overview. Following stress administration and sacrifice, the cellular morphology of astrocytes was evaluated in mice.

FIG. 15 illustrates results of a Flicker Intervention Study for Chronic Unpredictable Stress.

FIG. 16A shows flicker intervention study in males.

FIG. 16B shows flicker intervention study in males.

FIG. 16C shows flicker intervention study in males.

FIG. 17A shows Flicker intervention study in females.

FIG. 17B shows Flicker intervention study in females.

FIG. 17C shows flicker intervention study for chronic unpredictable stress in females.

FIG. 18 shows a Male Morphology Summary.

FIG. 19 shows Female Morphology Summary.

FIG. 20 illustrates Astrocyte reactivity after Chronic Stress and Flicker.

FIG. 21 further shows chronic audiovisual flicker boosts resilience to stress.

FIG. 22 illustrates that microglia play a causal role in certain stress and anxiety-like behaviors and the effects of flicker.

FIGS. 23A and 23B illustrate that stress leads to frequency-, sex- and region-specific morphological shifts.

FIG. 24 illustrates sensory flicker intervention boosts resilience during maladaptive stress exposure.

### DETAILED DESCRIPTION OF ILLUSTRATIVE EMBODIMENTS

The presently disclosed inventive subject matter may be understood more readily by reference to the following detailed description taken in connection with the accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a treatment” is a reference to one or more of such treatments and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to +10% of the recited value, inclusive. For example, the phrase “about 8” preferably refers to a value of 7.2 to 8.8, inclusive; as another example, the phrase “about 8%” preferably refers to a value of 7.2% to 8.8%, inclusive. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.” The phrase “at least about x” is intended to embrace both “about x” and “at least x”. It is also understood that where a parameter range is provided, all integers within that range, and tenths thereof, are also provided by the invention. For example, “2-5 hours” includes 2 hours, 2.1 hours, 2.2 hours, 2.3 hours etc., up to 5 hours.

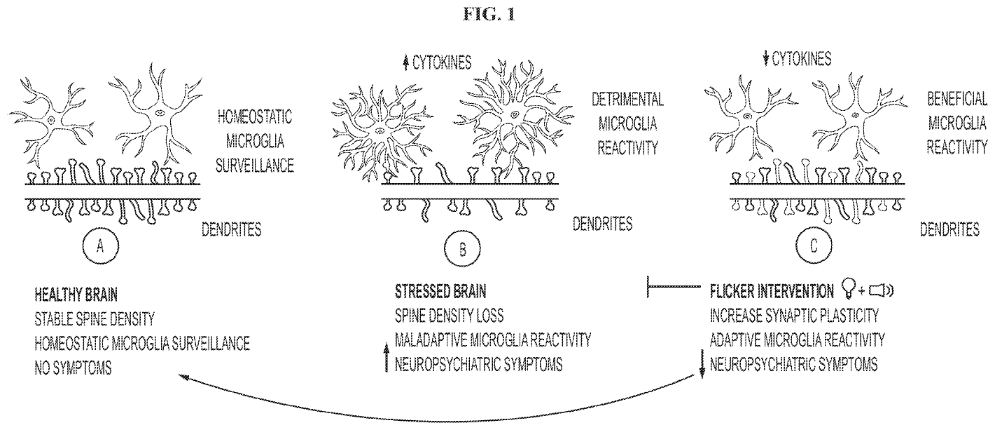

One of the primary mechanisms by which stress is thought to increase risk for neurodegenerative and neuropsychiatric disease is by inducing synapse loss and dysfunction through the dysregulation of inflammatory signaling. Chronic and severe stress promotes the overproduction of proinflammatory cytokines in the brain through persistent activation of microglia, the primary immune cells of the brain (FIG. 1, middle).^{10,37} Growing evidence suggests that stress enhances risk for dementia, depression, and anxiety disorders via immune dysregulation. This is thought to occur in part through neuroimmune inflammatory complement cascades that drive phagocytosis of synaptic connections on spines and dendritic arbors through classical complement cascade signaling pathway. Classical complement cascade activation in glia cells, such as microglia and astrocytes, mediate the tagging of vulnerable synaptic elements to select and improve synaptic connections during development and learning and memory and removal of cellular debris throughout life. However, maladaptive re-activation of complement cascade signaling pathways under chronic or severe stress conditions results in excessive microglia mediated engulfment of synaptic elements and promotes the onset and progression of neuropsychiatric and neurodegenerative disorders. While transient activation of microglia can be beneficial (e.g., to engulf and clear pathogens), chronic stress leads to the dysfunctional and persistent activation of microglia, which in turn leads to excessive inflammatory signaling and synaptic pruning.

Provided herein are methods for increasing resilience to stress-induced pathology in a subject comprising exposing the subject to brain stimulation that is effective to drive oscillations, rhythmic electrical activity, or both in the subject at a frequency of about 5-100 Hz, wherein the subject is not currently subject to a diagnosis of a neurological or neuropsychiatric disorder, and, wherein the subject is selected for exposure to the brain stimulation based on (i) the subject's previous exposure to a stressor, (ii) the subject's contemporaneous exposure to a stressor, or (iii) an anticipated exposure of the subject to a future stressor.

As noted, chronic stress leads to a two-fold or more increased risk for multiple psychiatric and neurological disorders including AD and major depressive disorder (MDD). The failures of many AD treatments are thought to be due to intervening too late in the disease progression, therefore preventative treatments are sorely needed. Chronic stress increases the risk of developing AD at a similar rate (2-fold or more) as well-studied genetic risk factors like apoE4 (a single copy of apoe4 increases risk by two to three-fold) and chronic stress increases neuropsychiatric symptoms like anxiety and anhedonia, which often appear early in AD. Stress-induced changes, including anxiety and anhedonia behaviors, synaptic loss, and microglial synapse elimination also represent risk factors for the development of other stress related diseases including depression and anxiety disorders, as well as a host of other neuropsychiatric and neurodegenerative diseases (FIG. 1, middle).

The present inventors have discovered that non-invasive neural stimulation differentially modulates brain signals, including brain immune signals, in a frequency-specific manner, recruits microglia while preserving synaptic density, and beneficially affects the human neuroimmune system. Hence, the presently disclosed methods increase resilience to stress-induced pathology in a subject that, at the time of treatment, is not subject to a diagnosis of a neurological or neuropsychiatric disorder, but is selected for exposure to the brain stimulation based on (i) the subject's previous exposure to a stressor, (ii) the subject's contemporaneous exposure to a stressor, or (iii) an anticipated exposure of the subject to a future stressor, wherein the stressor could otherwise present the risk of causing stress-induced changes that could lead to the development of stress pathology. As used herein, “stress pathology” refers to neuronal, endocrine, or immunological modulation within a subject that represent biological features of neuropsychiatric or neurodegenerative disease. Thus, the present methods reduce susceptibility to neuropsychiatric or neurodegenerative disease by increasing resistance to stressors and stress pathology.

An important feature of the present methods is their use in connection with subjects that are not currently subject to a diagnosis of a neurological or neuropsychiatric disorder. For example, the subject may be one who is not currently subject to a diagnosis of a neurodegenerative disease. Previous studies have assessed the effect of brain stimulation on AD pathology. Flicker mediated changes in microglia function has been shown to coincide with the preservation of synaptic density and increased expression of synapse-associated markers in male mouse models of neurodegeneration. In a feasibility study, there was excellent safety and adherence to daily gamma stimulation over 4-8 weeks in AD patients at home, and gamma sensory stimulation over 8 weeks altered cytokine levels in the cerebral spinal fluid (CSF), indicating that gamma stimulation may modulate the human immune system in AD patients. It was not known whether brain stimulation could increase resilience to stress-induced pathology, i.e., reduce susceptibility to neuropsychiatric or neurodegenerative disease in the first instance by increasing resistance to stressors that could otherwise lead to pathological neuronal, endocrine, or immunological modulation. The present methods, in contrast to previous approaches, have beneficial application with respect to subjects that have had a previous exposure to a stressor, are being exposed to a stressor contemporaneously with stimulation in accordance with the inventive methods, or are anticipated to be exposed to a stressor in the future. The present methods may also be used with respect to subjects that are in remission from a stress-induced pathology, or are in remission from a neuropsychiatric disorder.

The stressor may be of any variety, and may include, for example, a personal loss of a friend, loved one, or property (such as a home), a challenging family environment or situation (such as a problem with a spouse, former spouse, parent, or child, or even a childbirth), physical or emotional abuse, victimization by criminal activity, a difficult or demanding occupation, financial difficulty, or an automobile or other accident or personal injury. The stressor may be acute, i.e., may occur over a limited period of time, or may be something that is experienced repeatedly, over an extended period of time (such as multiple days, weeks, or months), or both repeatedly and over an extended period of time. A single stressor, or more than one type of stressor, may be at issue. A clinician may be made aware of the stressor based on reporting directly from the subject, the subject may have been referred for treatment based on the recommendation of a third party, or the subject may be self-referred.

In some embodiments, the subject is selected for exposure to the brain stimulation based on the subject's exposure to a prior stressor. The stressor may have occurred days, weeks, a month, more than one month, a year, or more than one year prior to the exposure by the subject to brain stimulation.

The subject may alternatively be selected for treatment based on the subject's exposure to a stressor contemporaneously with the treatment. In other words, the subject may be experiencing the stressor on an ongoing basis at the time of selection for treatment.

In other embodiments, the subject is selected for exposure to the brain stimulation based on an anticipated exposure of the subject to a future stressor. Thus, the subject can be selected for treatment it can be ascertained that the subject will or is likely to be exposed to a stressor sometime at a point in the future relative to the contemplated treatment. An exemplary situation is one in which the subject is planning on a stressful personal change or event, such as a difficult or demanding occupation, an anticipated personal loss (such as when a loved one is nearing death), or a planned life change, such as a divorce, family separation (for example, due to military deployment), or physical relocation. The stressor may be anticipated to occur days, weeks, a month, more than one month, a year, or more than one year following the time of contemplated exposure to the brain stimulation.

Accordingly, the subject may be selected for treatment by exposure to the brain stimulation by assessing the subject's susceptibility to stress-induced pathology. This may involve a determination of a previous, ongoing, or anticipated stressor. In some embodiments, the assessment of the subject's susceptibility to stress-induced pathology can include a self-perceived stress assessment, an anxiety assessment, an anhedonia assessment, a measurement of one or more biomarkers of stress pathology in the subject, or a combination thereof. The assessment of the subject's susceptibility to stress-induced pathology may involve determining whether the subject has a history of one or more mood disorders (for example, if the subject is in remission at the time of the assessment), wherein a positive determination would result in a conclusion that the subject is at least potentially susceptible to stress-induced pathology. The assessment of the subject's susceptibility to stress-induced pathology may involve determining whether the subject has had a previous neuropsychiatric disorder, such as if the subject is in remission from a neuropsychiatric disorder.

The brain stimulation to which the subject is exposed pursuant to the present methods may be any type of stimulation that is effective to drive oscillations, rhythmic electrical activity, or both in the subject at a frequency of about 5-100 Hz. One type of brain stimulation that can be used for this purpose is audiovisual flicker. This type of stimulation involves flickering lights and sounds with millisecond precision. The brain stimulation may alternatively be electrical, such as by use of externally-positioned or internally-positioned electrodes, magnetic, or ultrasound.

Depending on whether the subject is male or female, the brain stimulation may be selected such that it drives oscillations, rhythmic electrical activity, or both at a particular frequency within the range of 5-45 Hz. Stress pathophysiology differs between the sexes, and the frequencies of stimulation that best promote resilience for males can be different than for females. As described infra in Example 1, a mouse model for assessing the effects of flicker on stress demonstrated that male mice benefitted from stimulation by different frequencies than for female mice. Accordingly, whether the subject is male or female can influence the selection of an appropriate frequency in the range of 5-100 Hz at which to drive oscillations, rhythmic electrical activity, or both. The present methods may therefore comprise selection of the frequency at which the oscillations, rhythmic electrical activity, or both in the subject are driven based on the subject's sex.

In certain embodiments, the frequency at which the oscillations, rhythmic electrical activity, or both in the subject are driven may be selected based on the particular stress-induced pathology to which the subject is susceptible. For example, if the subject has a genetic risk of developing a particular stress-induced pathology, then the frequency at which the oscillations, rhythmic electrical activity, or both in the subject are driven may be based thereon.

The frequency at which the oscillations, rhythmic electrical activity, or both in the subject are driven may be selected based on the identity of the region of the subject's brain for which brain stimulation is desired. For example, the frequency can be selected depending on whether the region of the brain for which brain stimulation is desired is the hippocampus (HPC), amygdala (AMY), prefrontal cortex (PFC), nucleus ccumbens (NAc) or any combination thereof. If the region of the brain is the HPC or PFC, it may be desirable to select a frequency of about 10 to 40 Hz. If the region of the brain is the amygdala, a different frequency may be selected. As described herein, beneficial effects have been observed from 10-40 Hz in HPC and PFC. Good response has been observed in PFC at 10 Hz in males and 40 Hz in females, with some beneficial effects at other frequencies.

The frequency at which the oscillations, rhythmic electrical activity, or both in the subject are driven may be selected based on two or more factors selected from, for example, the subject's sex, the region of the brain for which stimulation is desired, the particular stress-induced pathology to which the subject is susceptible, or another factor. For example, if the region of the brain for which stimulation is desired is the amygdala and the subject is male, then the selected frequency may be 40 Hz.

Generally speaking, the frequency or frequencies at which the oscillations, rhythmic electrical activity, or both in the subject are driven may be about 5-100 Hz. For example, the frequency may be about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Hz. Over the course of a treatment regimen, the subject may be exposed to brain stimulation that drives oscillations, rhythmic electrical activity, or both at a particular frequency or range of frequencies at certain points during the regimen, and at other points in time during the regimen, different frequencies or ranges of frequencies may be used. For example, the present methods embrace treatment regimens in which brain stimulation drives the relevant effects at about 20 Hz at certain points in time (e.g., on certain days), and at other points in time the brain stimulation drives the relevant effects at a different frequency or range of frequencies, such as at 30 Hz. As described more fully below, the selected frequency may be adjusted over time during the course of the present methods, in order to refine the treatment of the subject to ensure maximal efficacy in terms of promoting resilience to stress pathology.

The oscillations that are driven by the brain stimulation may be gamma oscillations, beta oscillations, theta oscillations, alpha oscillations, delta oscillations, or a combination thereof.

In order to drive the oscillations, rhythmic electrical activity, or both, at a frequency of about 5-100 Hz, the brain stimulation itself may occur at a frequency of about 5-100 Hz. For example, the frequency of the applied brain stimulation may be about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Hz. For example, when audiovisual flicker represents the brain stimulation, the audiovisual flicker may occur at frequency of about 5-100 Hz. In certain embodiments, the brain stimulation is audiovisual flicker that occurs at a frequency of about 20 Hz.

The duration of a particular session of brain stimulation, the frequency (in the sense of rate of recurrence) of sessions, for example, over the course of a week, and the duration of a treatment regimen that involves the brain stimulation may be selected in order to provide the highest degree of benefit to the subject. For example, the duration of a particular session of brain stimulation may be about 5 minutes to 2 hours. Thus, the duration of a particular session of brain stimulation may about 5, 10, 15, 20, 25, 30, 35, 40, 45, 50, 55, 60, 65, 70, 75, 80, 85, 90, 95, 100, 105, 110, 115, or 120 minutes. The subject may be exposed to a particular session of brain stimulation once or multiple times per day, every other day, or once every three, four, five, six, or 7 days. In some preferred embodiments, a treatment regimen may involve exposing the subject to a particular session of brain stimulation once per day. When it occurs once per day, particular session of brain stimulation may occur, for example, at approximately the same time each day, or within 1-6 hours of the same time each day. In some embodiments, exposure of the subject to the brain stimulation occurs at a relatively fixed time (e.g., within 1-6 hours of a particular time of day) on a daily basis. The duration of a treatment regimen that involves multiple individual sessions of the brain stimulation may be several days, a week, multiple weeks, a month, two months, or multiple months. In one example, the subject is exposed to the brain stimulation for about 15-90 minutes per day for seven or more days, such as for 7-30 days.

The present methods may further comprise assessing the efficacy of the brain stimulation in the subject following the exposure of the subject to the brain stimulation. The assessment may rely on any type of testing methodology that indicates the levels of stress, the degree of stress reduction, or both, following the brain stimulation. In some embodiments, the assessment includes anxiety testing. In some embodiments, the assessment may rely on self-testing, whereby the subject provides report concerning the level of stress being experienced by the subject himself or herself. In such instances, a perceived stress scale (PSS) or any other self-testing and reporting methodology may be utilized. The assessment of the efficacy of the brain stimulation may also or alternatively include measuring one or more biomarkers of stress-induced pathology in the subject. In this context, “biomarkers” can refer to any physiological indicators of stress, including, for example, cytokines, microglia activity, synaptic gene expression levels, endocrine-related markers such as cortisol levels (which can be assessed via saliva), circulating inflammatory markers, and including markers that are detectable by MRI or PET imaging. Biomarkers in this context can also refer to physiological responses to questionnaires (CRH), stress hormones like corticotropin-releasing hormone (CRH) and cortisol, activity and functional connectivity in the default mode network, measures of allostatic load like assessment of the hypothalamic-pituitary-adrenal (HPA) axis via serum dehydroepian-drosterone sulfate (DHEA-S), the sympathetic nervous system (urinary norepinephrine and epinephrine), the cardiovascular system (systolic and diastolic blood pressure, serum high-density lipoprotein (HDL) and total cholesterol concentrations), metabolic processes (plasma glycosylated hemoglobin, a measure of glucose levels over time), or skin conductance assessment to measure autonomic nervous system arousal.

The brain stimulation in accordance with the present methods increases resilience to stress pathology. In doing so, it can reduce the risk of or reduce the expression of behaviors that result from stress pathology, such as anxiety, depression, anhedonia, or decreased cognitive performance. More directly, the brain stimulation can increase resilience to stress-induced pathologies such as synaptic loss, neuronal atrophy, immune dysregulation, and, generally, neurodegenerative disease.

Some of the direct effects of the brain stimulation pursuant to the present methods that produce the resilience to stress pathology include an increase in synaptic marker expression, beneficial alteration in microglia activity (such as by reducing microglia-mediated synaptic pruning; other types of microglia modulation are described in the working examples, infra), and beneficial alteration in cytokine expression.

Also disclosed herein are methods for increasing resilience to stress-induced pathology in a subject comprising exposing the subject to brain stimulation that is effective to drive oscillations, rhythmic electrical activity, or both in the subject at a frequency of about 5-100 Hz, wherein the subject is not currently subject to a diagnosis of a neurological or neuropsychiatric disorder, and wherein the subject has been assessed as having an elevated genetic risk for a neurological or neuropsychiatric disorder. Thus, in contrast with the previously described methods, the subject need not have been selected for the brain stimulation based on a past, ongoing, or anticipated future stressor. Instead, the subject is selected for the brain stimulation based on an assessment of genetic risk for a neurological or neuropsychiatric disorder, even if no particular stressor is at issue. The other aspects of the present methods may be consistent with any of the above-describe embodiments of the other disclosed methods, including the characteristics of the brain stimulation, the factors that influence the selection of the characteristics of the brain stimulation, and the optional assessment of the efficacy of the brain stimulation following an exposure of the subject to the brain stimulation, the physiological effects of the brain stimulation. Accordingly, to the extent that they are applicable, any of the previously described embodiments can be used connection with the current methods involving a subject having a genetic risk for a neurological or neuropsychiatric disorder.

The genetic risk for the neurological or neuropsychiatric disorder can be assessed using the subject's family history (such as if one or more close relatives suffered from a neurological or neuropsychiatric disorder, and the disorder has a genetic link), or the subject expresses known genetic risk factors for the neurological and neuropsychiatric disorder, like apoE, inflammatory, classic complement, or microglia genes.

The neurological or neuropsychiatric disorder for which the subject has a genetic risk may be, for example, Alzheimer's disease, depression, anxiety, schizophrenia, or autism. In some embodiments, the neurological or neuropsychiatric disorder for which the subject has a genetic risk may be related to classic complement signaling. Examples of such disorders include bipolar disorder, amyotrophic lateral sclerosis, Parkinson's disease, attention deficit-hyperactivity disorder, obsessive-compulsive disorder, multiple sclerosis, systemic lupus, autism, inflammatory bowel disease, type-2 diabetes, and age-related macular degeneration. For purposes of the present disclosure, the “neurological or neuropsychiatric disorder” may embrace quasi-disorders and other neurological states, such as migraines and seizure disorders, including epilepsy.

Also disclosed herein are methods for treating a neurological or neuropsychiatric disorder in a subject for whom an antidepressant or anti-anxiety medication is contraindicated or who is resistant to treatment with an antidepressant or anti-anxiety medication comprising exposing the subject to brain stimulation that is effective to drive oscillations, rhythmic electrical activity, or both in the subject at a frequency of about 5-100 Hz. Pursuant to such methods the subject need not have been selected for the brain stimulation based on a past, ongoing, or anticipated future stressor. Instead, the subject is selected for the brain stimulation based on a contraindication for an antidepressant or anti-anxiety medication or resistance to treatment with an antidepressant or anti-anxiety medication, even if no particular stressor is at issue. The other aspects of the present methods may be consistent with any of the above-describe embodiments of the other disclosed methods, including the characteristics of the brain stimulation, the factors that influence the selection of the characteristics of the brain stimulation, and the optional assessment of the efficacy of the brain stimulation following an exposure of the subject to the brain stimulation, the physiological effects of the brain stimulation. Accordingly, to the extent that they are applicable, any of the previously described embodiments can be used connection with the current methods involving a subject having a contraindication or resistance to treatment with antidepressant or anti-anxiety medication.

The contraindication for treatment with antidepressant or anti-anxiety medication can be due to an underlying physical condition or the fact that the subject engaged in a course of treatment with a contraindicated medication. Subjects for whom treatment with antidepressant or anti-anxiety medication is contraindicated may include, for example, pregnant or nursing women. Antidepressants should be used with caution in patients with known hypersensitivities or who are taking other psychotropic medications. Selective serotonin reuptake inhibitors (SSRIs) and serotonin-norepinephrine reuptake inhibitors (SNRIs), for example, should not be taken with other SSRIs, monoamine oxidase inhibitors, tricyclic antidepressants, and other psychotropics; this is due to the risk of serotonin syndrome, which can lead to severe neuromuscular and autonomic symptoms. Tricyclic antidepressants can provide another good example of relative contraindications in antidepressant therapy. Clinicians should be mindful when prescribing tricyclic antidepressants to individuals with cardiovascular disease. Tricyclic antidepressants have been shown to cause orthostatic hypotension. Additionally, in patients with preexisting bundle-branch disease, tricyclic antidepressants may lead to heart block. Buproprion, an atypical antidepressant, has seizure disorder listed as a major contraindication. This contraindication applies to patients with an active seizure diagnosis or with a history of prior seizure activity. Like other antidepressants, bupropion should not be used in patients taking monoamine oxidase inhibitors or drugs that can lower the seizure threshold. Liver injury due to previous treatment is a contraindication to nefazodone therapy. Esketamine is contraindicated in aneurysmal vascular disease (thoracic and abdominal aorta, intracranial, and peripheral arterial vessels), arteriovenous malformations according to the product labeling. The present methods are intended to embrace any type of contraindication that makes treatment with antidepressant or anti-anxiety medication, or treatment with antidepressant or anti-anxiety medication at a particular dosage, wherein such dosage is otherwise required for efficacious treatment, unadvisable or undesirable for a subject, but for whom some form of alternative or supplemental treatment is desirable.

A subject's resistance to treatment with antidepressant or anti-anxiety medication can be partial or complete. In other words, the subject may be unresponsive to such treatment or may evince an unsatisfactorily low response to treatment with antidepressant or anti-anxiety medication. The present methods are intended to embrace all such scenarios. Accordingly, the present methods can be supplemental to treatment of the subject with antidepressant or anti-anxiety medication, or can be used for subjects for whom treatment with antidepressant or anti-anxiety medication has been ceased or suspended.

Also disclosed herein are methods for treating dysregulation of classic complement signaling, dysregulation of microglia activity, neuroinflammation, or pathological synaptic change in a subject comprising exposing the subject to brain stimulation that is effective to drive oscillations, rhythmic electrical activity, or both in the subject at a frequency of about 5-100 Hz, wherein the subject is selected for exposure to the brain stimulation based on a determination of neuroinflammation, dysregulation of classic complement signaling, dysregulation of microglia activity, or pathological synaptic change in the subject, or, wherein the subject is selected for exposure to the brain stimulation based on a determination that the subject has an elevated genetic risk for a condition having a classic complement component.

As used throughout the present disclosure, “treating” or “treatment” can refer to the at least partial amelioration of the relevant state or condition, such that the subject receiving the treatment becomes closer to a healthy (non-pathological) state with respect to the pertinent state or condition. For example, treatment of a parameter can refer to at least partial normalization with respect to the relevant parameter. Treatment can also or alternatively refer to reduction of dysregulation or disfunction with respect to the parameter.

The dysregulation of classic complement signaling may characterized by elevated classic complement signaling relative to a reference value corresponding to a normal level of classic complement signaling. In other embodiments, the dysregulation of classic complement signaling is characterized by decreased classic complement signaling relative to a reference value corresponding to a normal level of classic complement signaling. The reference value may be determined using measurement of classic complement signaling from a subject in whom it is known that normal levels of classic complement signaling are present, may be determined from a population of subjects in each of whom it is known that normal levels of classic complement signaling are present, or may otherwise be calculated using according to accepted methodologies.

In other embodiments, dysregulation of classic complement signaling may characterized by a measured amount classic complement signaling that is not statistically different from a reference value corresponding to an elevated level of classic complement signaling. The reference value may be determined using measurement of classic complement signaling from a subject in whom it is known that elevated levels of classic complement signaling are present, may be determined from a population of subjects in each of whom it is known that elevated levels of classic complement signaling are present, or may otherwise be calculated using according to accepted methodologies.

In other embodiments, dysregulation of classic complement signaling may characterized by a measured amount classic complement signaling that is not statistically different from a reference value corresponding to a decreased level of classic complement signaling. The reference value may be determined using measurement of classic complement signaling from a subject in whom it is known that decreased levels of classic complement signaling are present, may be determined from a population of subjects in each of whom it is known that decreased levels of classic complement signaling are present, or may otherwise be calculated using according to accepted methodologies.

Dysregulation of microglia activity may be characterized by elevated microglia activity relative to a reference value corresponding to a normal level of microglia activity. In other embodiments, dysregulation of microglia activity may be characterized by decreased microglia activity relative to a reference value corresponding to a normal level of microglia activity. The reference value may be determined using measurement of microglia activity from a subject in whom it is known that normal levels of microglia activity are present, may be determined from a population of subjects in each of whom it is known that normal levels of microglia activity are present, or may otherwise be calculated using according to accepted methodologies.

In other embodiments, dysregulation of microglia activity may be characterized by a measured amount microglia activity that is not statistically different from a reference value corresponding to an elevated level of microglia activity. The reference value may be determined using measurement of microglia activity from a subject in whom it is known that elevated levels of microglia activity are present, may be determined from a population of subjects in each of whom it is known that elevated levels of microglia activity are present, or may otherwise be calculated using according to accepted methodologies.

In other embodiments, dysregulation of microglia activity may be characterized by a measured amount microglia activity that is not statistically different from a reference value corresponding to a decreased level of microglia activity. The reference value may be determined using measurement of microglia activity from a subject in whom it is known that decreased levels of microglia activity are present, may be determined from a population of subjects in each of whom it is known that decreased levels of microglia activity are present, or may otherwise be calculated using according to accepted methodologies.

Synaptic loss or dysfunction in a subject may characterized, for example, by decreased brain volume, synaptic density in the brain, or change in synaptic marker expression relative to a reference value corresponding to a normal volume level or normal level of synaptic marker expression. The reference value may be determined using measurement of synaptic loss or dysfunction from a subject in whom it is known that normal levels of volume, synaptic density or synaptic markers are present, may be determined from a population of subjects in each of whom it is known that normal levels of volume, synaptic density, or synaptic markers are present, or may otherwise be calculated using according to accepted methodologies.

As used in the present disclosure, “pathological synaptic change” refers to any regional net loss or net gain of synaptic connections relative to a healthy synaptic state, and/or any regional change in synaptic strength relative to a healthy synaptic state, such as that resulting from a stressor or other neurological trauma. In certain embodiments, the presence of pathological synaptic change in the subject may characterized by a measured amount of volume, synaptic density, or synaptic markers that is not statistically different from a reference value corresponding to pathological synaptic change. The reference value may be determined using measurement of synaptic change from a subject in whom it is known that there is pathological synaptic change, may be determined from a population of subjects in each of whom it is known that there is pathological synaptic change, or may otherwise be calculated using according to accepted methodologies.

In accordance with certain embodiments, the subject may selected for exposure to the brain stimulation based on a determination that the subject has an elevated genetic risk for bipolar disorder, amyotrophic lateral sclerosis, Parkinson's disease, attention deficit-hyperactivity disorder, obsessive-compulsive disorder, multiple sclerosis, systemic lupus, autism, inflammatory bowel disease, type-2 diabetes, or age-related macular degeneration. The genetic risk for the condition having a classic complement component can be assessed using the subject's family history (such as if one or more close relatives suffered from a condition having a classic complement component, and the condition has a genetic link), or the subject expresses known genetic risk factors for the condition, like apoE, inflammatory, classic complement, or microglia genes.

The other aspects of the present methods for reducing dysregulation of classic complement signaling, dysregulation of microglia activity, neuroinflammation, or pathological synaptic change in a subject may be consistent with any of the above-describe embodiments of the other disclosed methods, including the characteristics of the brain stimulation, the factors that influence the selection of the characteristics of the brain stimulation, and the optional assessment of the efficacy of the brain stimulation following an exposure of the subject to the brain stimulation, the physiological effects of the brain stimulation. Accordingly, to the extent that they are applicable, any of the previously described embodiments can be used connection with the current methods for reducing dysregulation of classic complement signaling, dysregulation of microglia activity, neuroinflammation, or pathological synaptic change in a subject.

### EXAMPLES

The present invention is further defined in the following Examples. It should be understood that these examples, while indicating preferred embodiments of the invention, are given by way of illustration only, and should not be construed as limiting the appended claims. From the above discussion and these examples, one skilled in the art can ascertain the essential characteristics of this invention, and without departing from the spirit and scope thereof, can make various changes and modifications of the invention to adapt it to various usages and conditions.

### Example 1-Flicker Induces Behavioral Resilience to Chronic Stress

Chronic stress increases the risk of Alzheimer's disease (AD) and anxiety and depressive disorders. Furthermore, chronic stress accelerates the development of neuropsychiatric symptoms and cognitive decline.^{17,98-102} Previous studies have established that gamma sensory flicker improves cognitive deficits in AD mice.^{27,28} However, the effects of sensory flicker on stress-induced behavioral deficits in health or AD has never been explored. The present inventors investigated whether flicker intervention during chronic stress exposure will protect against neuropsychiatric deterioration and cognitive deficits in healthy mice and protect against accelerated cognitive and neuropsychiatric deterioration in 5XFAD mice and apoE4-KI mice.

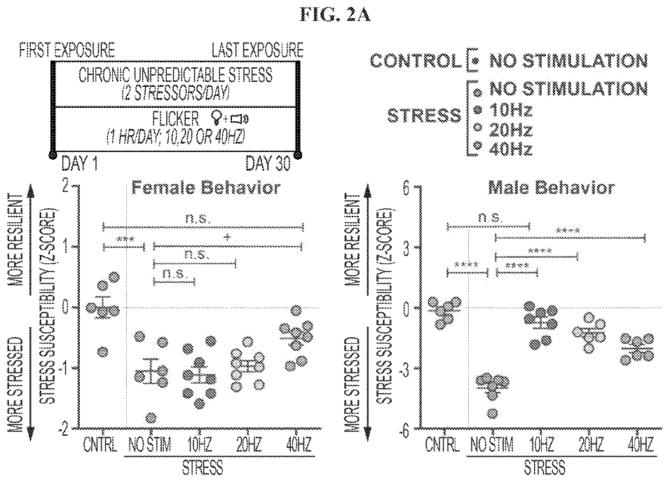

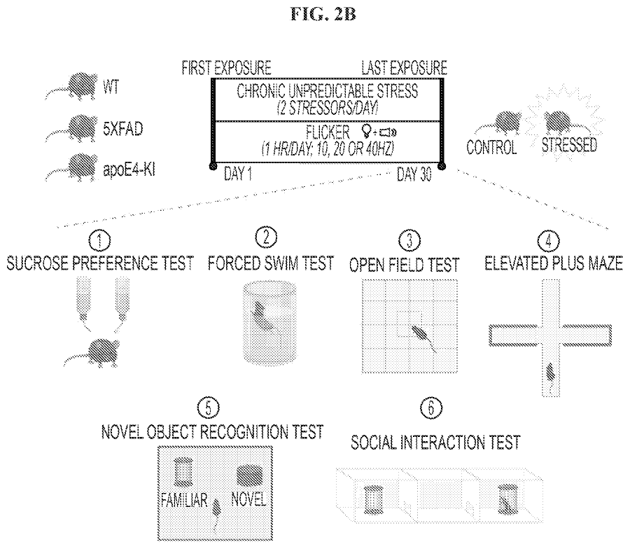

It was found that sensory flicker promotes resilience to stress at the behavioral, cellular, and molecular levels. The experimental data demonstrated that chronic multi-sensory flicker leads to increased resilience to stress at the behavioral level in a sex- and frequency-specific manner (FIG. 2A). After animals underwent 21 days of chronic audio-visual flicker exposure and CUS, a battery of behavioral tests was performed to assess anxiety, anhedonia, general activity levels, and memory including the open field, novel object recognition, sucrose preference, elevated plus maze, and forced swim tests. The results of these assays were combined into a composite stress susceptibility score that indicates more stressed or more resilient phenotypes across the entire battery of tests relative to no stress, no stimulation control animals (FIG. 2A).¹⁰³ The effects of stress and flicker were examined in both male and female mice because the effects of stress differ between sexes.¹⁰⁴⁻¹⁰⁸

FIG. 2A depicts how adult male mice underwent either no stress and no flicker (control, grey), chronic unpredictable stress (CUS) alone (red), or CUS and 1 hr of flicker at 10 Hz (blue), 20 Hz (turquoise), or 40 Hz (green) for 1 hr/day for 30 days. From day 21 to 30, mice undergo open field, sucrose preference, novel object recognition, elevated plus maze, and forced swim tests. Results from each behavior were compared to no stress controls and combined into a composite stress susceptibility z-score that indicated more stressed (negative) or more resilient (positive) phenotypes. In FIG. 2A, bottom left, females that underwent CUS and 40 Hz flicker (green) exhibited less stressed behaviors than CUS alone (red) and were more similar to no stress control mice (grey) while 10 Hz and 20 Hz did not seem to have strong effects. In FIG. 2A, bottom right, males that underwent CUS and 10 Hz (blue) or 20 Hz (turquoise) flicker behaved similarly to no stress control mice (grey).

Behavior of males that underwent CUS and 40 Hz flicker (green) was in between no stress control (grey) and CUS alone mice (red). n.s. indicates not significant, ***p=0.0001, ****p<0.0001, +p=0.051.

Thus, it was found that stressed male mice that underwent 10 or 20 Hz flicker showed more resilient behavior responses than no stimulation stressed animals, and were similar to control animals, while stressed males that underwent 40 Hz flicker showed an intermediate response between no stimulation stressed animals and control animals. In contrast, stressed females that underwent 40 Hz flicker showed more resilient behavior, while stressed females that underwent 10 or 20 Hz flicker had similar behavioral responses to stressed mice. The preliminary results show that flicker boosts behavioral resilience in WT mice and the effects are sex and flicker frequency dependent.

### Example 2—Brain Stimulation to Prevent Stress-Induced Anxiety, Anhedonia, and Cognitive Defects in Sex-Specific Manner

Male and female wild-type (WT) and 5XFAD, and female apoE4-KI mice (N=10 per group per sex) are exposed to 30 days of CUS to induce neuropsychiatric-like behaviors and cognitive deterioration in WT and in prodromal stages in 5XFAD and apoE4-KI mice. Several studies have previously shown that CUS induces neuropsychiatric-like behaviors in WT mice and mouse models of amyloidosis. 6.37.94.109 During stress exposure, animals receive either chronic audio-visual flicker (light and sound at 10 Hz, 20 Hz or 40 Hz for 1 hour/day) treatment or no stimulation (no light/no sound) control. Mice are exposed to stressor and flicker on the same day at different times to mimic how individuals already undergoing stress (e.g., ER nurses, pilots) would be treated with flicker in conjunction with stress but prior to the onset of AD or in the prodromal phases of AD. A battery of behavior tests are conducted during the last seven days including assessment of cognitive deterioration in the novel object recognition and object location tests, assessment of anhedonia with sucrose preference test and social interaction test, and anxiety and despair-like behaviors in the elevated plus maze and forced swim test, respectively (FIG. 2B). The number of animals required for each experiment is determined using power analysis for differences determined from preliminary studies (power=0.80 and alpha=0.05) and from published studies (N=10 per group per sex).^{37,109}

As shown in FIG. 2B, adult male and female WT and 5XFAD mice and female apoE4-KI mice undergo either no stress and no flicker, chronic unpredictable stress (CUS) alone, or CUS and 1 hr of flicker at 10, 20, or 40 Hz for 1 hr/day for 30 days (N=10 mice per group per sex). From day 21 to 30, mice undergo (1) sucrose preference, (2), forced swim test, (3) open field, (4) elevated plus maze, (5) novel object recognition, and (6) social interaction tests to assess anxiety, anhedonia, and cognition. Results from each behavior are compared to no stress controls and combined into a composite stress susceptibility z-score that indicates more stressed or more resilient phenotypes.

The results of behavioral assays are combined into a composite stress susceptibility score that indicates more stressed or more resilient phenotypes across the entire battery of tests relative to no stress, no stimulation control animals (FIGS. 2A, 2B). Briefly, individual stress susceptibility scores are obtained by generating individual z scores normalized to controls from each sex for each performed behavior test. An overall susceptibility score is then generated by averaging individual z scores across all tests. Stress susceptibility set point is set at zero and negative scores are considered stressed for this set of selected behavioral tests and positive scores are considered stress resilient. This approach addresses the diversity of neuropsychiatric symptoms observed in patients with dementia and detects subtle changes in stress susceptibility between sexes or individuals.^{17,98-101} Quantification of behavioral changes after stress and flicker exposure is performed by two-way ANOVA (genotype x treatment) for each sex to determine if flicker leads to a significant change in stress susceptibility scores. Statistical significance is determined by a main effect (F-score) of p<0.05.

### Example 3—Brain Stimulation Promotes Resilience by Increasing Synapses and Preventing Synapse Loss Following Chronic Stress

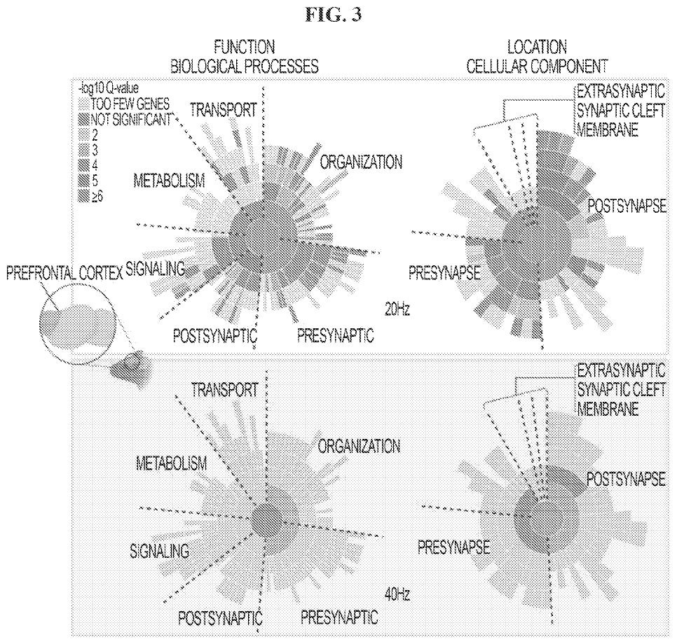

Growing evidence shows that chronic stress leads to functional and structural synaptic changes in corticolimbic brain regions in a sexual dimorphic manner, leading to sex-specific symptoms in stress-related diseases and AD.^{90,92,93,95-97,110-112} Synaptic loss in prefrontal cortex (PFC) and hippocampus (HPC) are predictive of neuropsychiatric and cognitive decline in AD, respectively.¹¹³⁻¹²⁰ The present data demonstrates that flicker promotes functional and structural gene expression increases at the synapse in a frequency-dependent manner (FIG. 3). As provided in FIG. 3, prolonged 20 Hz (top) or 40 Hz (bottom) audiovisual flicker for 1 hr/day for 21 days resulted in significant upregulation of synaptic genes in PFC with more pronounced effects after 20 Hz flicker. Concentric rings of sunburst plots, represent hierarchical structures with distance from center indicating distance from core genes. N=3 mice/group with 3 technical replicates.

However, the use of noninvasive non-pharmacological sensory flicker intervention as a modulator of synaptic loss has never been studied in the context of stress. Accordingly, the inventors' objective was to examine how flicker modulates synaptic loss in the context of stress in PFC and HPC. Microglia play a pivotal role in synapse formation in health and disease by selectively pruning synapses.^{8,62,121} Reduction of synaptic marker expression due to microglia's excessive pruning of synaptic material results in functionally relevant changes in synaptic transmission.⁶³ Moreover, studies demonstrate that inhibition of microglia mediated synaptic pruning rescues synaptic loss and dysfunction in AD associated brain regions.⁶³ Chronic stress leads to excessive microglia-mediated pruning of synaptic material which exacerbate risk for AD and depression and anxiety by increasing microglia-mediated synapse loss in vulnerable individuals.^{7-9,65} For example, older stressed rodents show more pronounced correlation between stress-induced microglia reactivity and spine density loss in the PFC when compared to younger stressed adult rodents.⁹ Although stress-induced spine density loss has been shown to be partially reversible following a period of stress recovery (absence of stress) in the subregions of PFC, stress-induced microglia reactivity has been shown to persist for several weeks throughout the PFC and HPC in rodents.^{37,122,123} Flicker has been shown to shift microglia responses in models of AD to increase microglia phagocytosis of Aβ in the PFC and HPC.^{26,27} Flicker mediated changes in microglial function has been shown to coincide with the preservation of synaptic density and increased expression of synapse-associated markers in male mouse models of neurodegeneration.²⁸ Flicker may protect synapses by promoting adaptive microglial responses depending on the presence of pathological signals. However, it remains unclear if flicker shifts microglia responses to an adaptive state following chronic stress or if flicker could induce microglia phagocytosis of synapses.

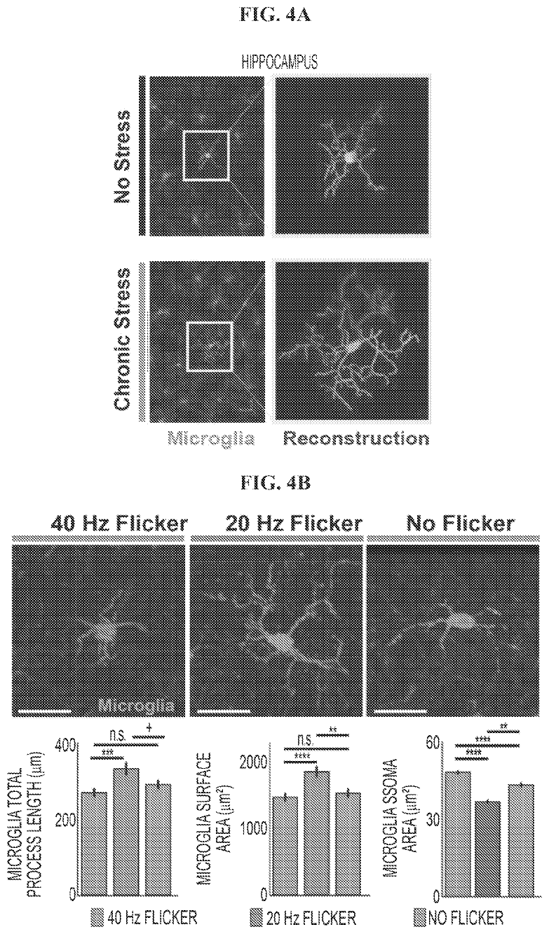

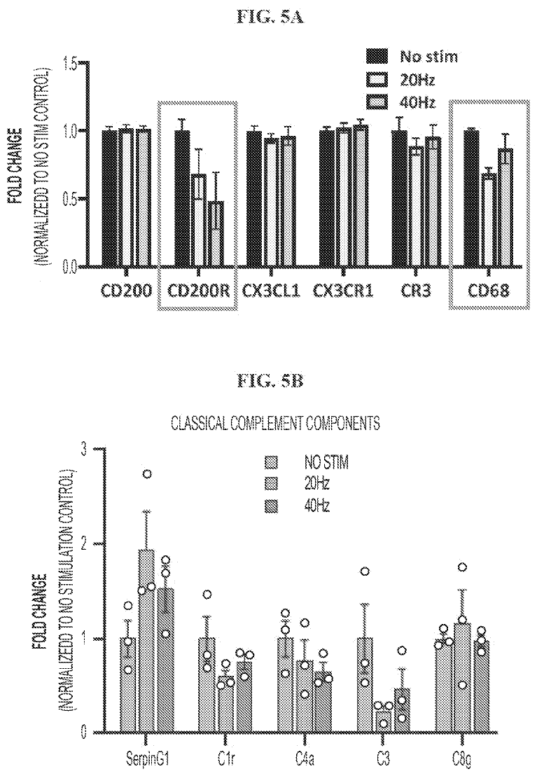

The present inventors have previously shown that multi-sensory audio-visual flicker at 40 Hz modulates firing of single neurons in stress-susceptible corticolimbic brain regions including the HPC and PFC.²⁷ Flicker may induce spiking synchrony increase synaptic plasticity in the corticolimbic system to reverse stress-induced synaptic functional and structural changes, as treatments for stress disorders have been shown to do. The inventors' histological data shows that flicker alters microglia morphology in a frequency-dependent manner (FIG. 4B). The inventors' transcriptomic data shows that chronic multi-sensory flicker alters synaptogenesis signaling pathways and microglia phagocytosis in a frequency-dependent manner. An increase in gene expression of synaptic markers was observed (including genes implicated in molecular signatures of depression such as SYNPR, SCG2 and GABRA1) in the PFC of wildtype male mice after chronic (1 hr/day for 21 days) audio-visual flicker exposure at 20 Hz (FIG. 3).¹²⁴ This effect was also observed at 40 Hz, although the effect was more subtle (FIG. 3). Furthermore, it was shown that multi-sensory flicker exposure in healthy C57BL/6 male mice activates microglia in the PFC (determined by shifted CD200R expression) while suppressing the expression of microglia phagocytosis marker CD68 in a frequency-dependent manner (FIG. 5A). FIG. 5 illustrates how prolonged audio-visual flicker (20H vs 40 Hz) reduced CD68 expression in the prefrontal cortex in a frequency-dependent manner and increased microglia activation as indicated by the reduction of CD200R expression (N=3 mice per group with 3 technical replicates). Notably, non-invasive modulation of cortical neural processing by transcranial magnetic stimulation (TMS) at 10 or 20 Hz has shown promising effects for the treatment of stress-related disorders including MDD and AD.¹²⁵ Unlike TMS, non-invasive flicker stimulation effectively entrains neurons in cortical regions and deeper brain structures associated with neuropsychiatric and cognitive symptoms.^{27,28} In addition, TMS appears to have modest effects on non-neuronal cells while flicker has been shown to robustly shift microglia dysfunction to beneficial responses in models of AD.^{27,126,127} This is particularly important for risk-modifying interventions of stress. Furthermore, chronic multi-sensory flicker suppresses the complement cascade, a “eat me” signal that tags synapses for engulfment by microglia. Excessive classic complement signaling has been shown to cause neuropsychiatric-like behaviors in mice and exacerbate AD pathology. The inventors found that chronic multi-sensory flicker upregulates expression of Serping1 (C1NH), the primary inhibitor of the classic complement initiator C1q complex⁴⁸, while downregulating expression of downstream complement effectors (FIG. 5B). Prolonged (1 hr/day for 21 days) 20 Hz (purple) and 40 Hz (green) A-V flicker leads to robust upregulation of classic complement inhibitor Serping 1 while downregulating complement component expression compared to no stimulation (grey) in prefrontal cortex of male mice.

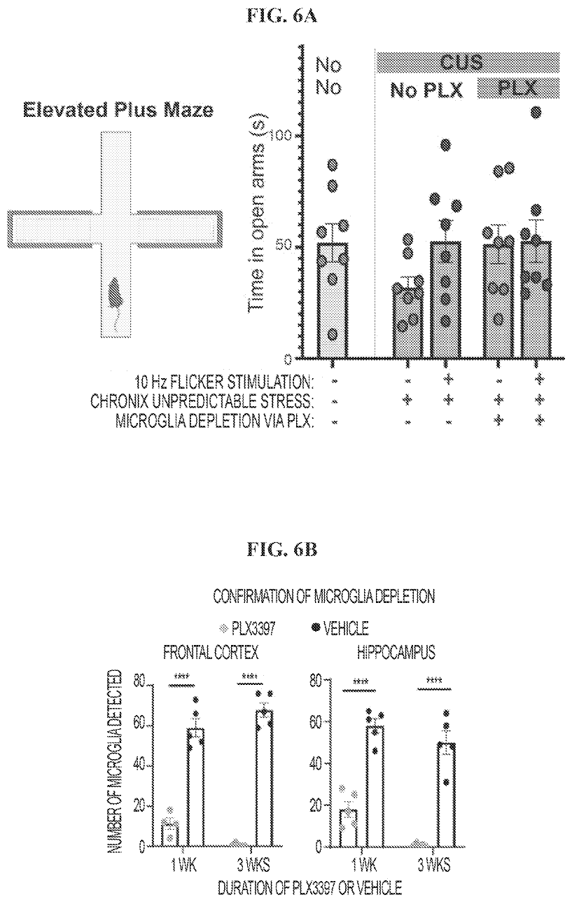

Finally, the present findings indicated that microglia are required for the effects of stress on anxiety behaviors and flicker did not further reduce stress effects without microglia (FIG. 6). In FIG. 6A, male stressed mice spent less time in open arms of the plus maze (left red bar) and this stress effect was reduced in stressed animals administered PLX to deplete microglia (right red bar). Flicker stimulation at 10 Hz mitigated the effects of stress (left blue bar) and flicker had no effect beyond that of PLX administration (right blue bar). Thus, this measure of anxiety-like behavior following stress was microglia dependent and flicker did not further reduce stress effects without microglia. These results suggest flicker effects this stress-induced behavior via microglia. In FIG. 6B, PLX application depleted microglia as indicated by significantly fewer microglia in frontal cortex and hippocampus after PLX (grey circles) compared to vehicle (black circles) after both 1-week and 3-weeks of PLX administration. n.s.=not significant, *p=0.0225, **p=0.0068, ****p<0.00005. The effects of brain stimulation is expected to be microglia-dependent when 40 Hz stimulation produces resilience, as it was observed in females, revealing different mechanism depending on sex (FIG. 2A).

### Example 4-Additional Investigation Concerning Resilience to Chronic Stress, and Changes in Synaptogenesis Signaling Pathways Following Stress and Brain Stimulation Intervention

To further test the hypothesis that brain stimulation promotes resilience by preventing synapse loss following chronic stress, tissue from WT, 5XFAD, and apoE4-KI animals that undergo stress and flicker, stress alone, or no stress and no stimulation (control) are assessed for synaptic markers and immune genes and proteins. This approach allows quantification of a large set of inflammation initiators, proinflammatory, anti-inflammatory and phagocytosis related proteins which are used to correlate synaptic gene expression changes with alterations in inflammatory signaling. Whole tissue is collected from PFC and HPC. Right hemisphere tissue is used for RNA sequencing (RNAseq) analysis and left hemisphere tissue is used for Olink inflammatory panel detection of markers associated with phagocytosis analysis for an integrative within subject analysis of transcriptomic and proteomic changes resulting from flicker exposure in the context of stress (N=10/group).

Both proteomics and transcriptomics are performed because, while transcriptomics provides a more comprehensive analysis of genetic effects, not all transcriptomic changes are propagated to proteins. For RNAseq analysis, samples are barcoded for multiplexing and sequenced at 100 bp paired-end on Illumina HiSeq2500 at the Georgia Institute of Technology Molecular Evolution Core. For Olink protein analysis, Olink Explore 384 Inflammation panel are used for an extensive quantification of inflammatory cytokines and phagocytosis proteins.^{29,128} This multiplex panel includes a wide range of proinflammatory, anti-inflammatory and phagocytosis related proteins that permit the correlation of synaptic gene expression changes with alterations in inflammatory signaling.

Analysis. Variance of gene expression and protein expression are performed separately using limma package of Bioconductor in R to identify differentially expressed genes (DEGs) and differentially expressed proteins (DEPs). Global gene ontology (GO; for global transcriptome and proteome classification) and Synaptic gene ontology (SynGO; for specific synaptic element classification) assessment are performed for DEGs and DEPs across brain regions in each sex separately. Rank-rank hypergeometric overlap (RRHO) test are performed to evaluate the degree of overlap in gene and protein signatures across brain regions in each sex separately as previously described for similar sex and region comparison studies.^{129,130} RRHO is also performed between sexes for each brain region.

To determine if microglia are required for the behavioral and synaptic effects of flicker, microglia are depleted using Csf1r inhibitor Pexidartinib (PLX3397, MedChem) incorporated into mouse chow at a dose of 290 mg/kg to deplete microglia and suppress monocytes. PLX3397 is a selective inhibitor of Csf1r that has been shown to rapidly deplete microglia by 70% after 7 days and ˜99% after 21 days when administered in mouse chow.¹³¹⁻¹³³ Mice are fed either the PLX Diet or the control diet (identical except without PLX) for 5 weeks. PLX treatment significantly decreased microglia presence after both one-week and three-week feeding durations (FIG. 6). After 1 week of chow, mice fed control and PLX chow are exposed to (1) no flicker and no CUS as a control, (2) CUS only, or (3) flicker stimulation and CUS for 4 weeks (N=10/group). Behavior assays are performed in the last week, and synaptic marker expression is assessed.

To assess synaptic spines and microglia engulfment of synapses, male and female Thy1-YFP H line mice are used to provide strong and specific Golgi-like labeling of excitatory neurons throughout the corticolimbic system in 5XFAD mice.⁶⁵ Thy1-YFP mice are transgenic reporter mice that have the yellow fluorescent gene expressed under the Thy1 gene, leading to the visualization of excitatory neurons with YFP. Both Thy1-YFP and 5XFAD crossbred with Thy1-YFP mice are used. Using this line facilitates the visualization of synaptic material phagocytosis by microglia in response to chronic stress as previously described in a similar line.⁶⁵ Male and female mice are exposed to 21 days of CUS to induce synaptic changes as previously shown. During stress exposure, animals receive either chronic audio-visual flicker (light and sound at 10 Hz or 40 Hz for 1 hour/day) treatment or no stimulation (no light/no sound) control as described in Aim 1. Mice are sacrificed by cardiac perfusion precisely 1 hour after the last flicker treatment and brain is collected for immunofluorescence (IF) analysis. Free-floating (30 μm) sections from medial PFC (2.68-1.54 mm Bregma) and dorsal HPC (−1.06-2.30 mm Bregma) are used to identify microglia by labeling for canonical microglia IBA1 marker. Subregions of the medial PFC (mPFC; prelimbic and infralimbic) and dorsal HPC (dHPC; CA1, CA3, DG) from both hemispheres are imaged by confocal microscopy from each selected section. Microglia density and morphology are assessed to quantify regional differences in the PFC and HPC following chronic stress (see, e.g., FIG. 4, N=6/group).^{26,37,134} The colocalization and inclusion of YFP″ material in IBA1-labeled microglia are used to measure microglia synaptic pruning and are used to determine if flicker intervention reverses stress-induced alterations in synaptic pruning. Separate (10 μm) tissue sections are stained and imaged via 2-photon microscopy and multi-color STORM superresolution Imaging (for the latter, STORM-validated secondary antibodies from Thermo Fisher Scientific are used).^{135,136} Because apical dendrites are particularly vulnerable to stress induced synaptic remodeling and spine density loss in the mPFC and dHPC in both males and females, distal apical dendritic arborization, branch length and spine density in the mPFC and dHPC are assessed in these samples. 90.95.137.138 For example, apical dendritic branch length and number (arbor) and dendritic spine density negatively correlate with stress susceptibility in the prefrontal cortex and dorsal hippocampus.^{39,42,139-144} Bilateral mPFC and dHPC images are analyzed via IMARIS or custom algorithms for 2-photon and STORM imaging, respectively, to provide a quantitative description of microglia-synapse interactions.^{39,42} STORM images are optimized to correct tissue aberrations and heterogeneity with Jia lab's point-spread function engineering techniques and adaptive optics.⁵¹ The number of animals required for each experiment is determined using power analysis for differences determined from preliminary studies (power=0.80 and alpha=0.05) and from published studies reporting significant sex-specific transcriptomic changes following stress.^{29,145}

Analysis. A linear mixed-model with fixed-effects are used for the intercept, treatment (no stimulation vs optimal frequency), region of interest (PFC, HPC) identity covariance structure, and maximum-likelihood estimation for each sex and genotype.

Analysis of molecular and cellular changes after stress and flicker exposure is performed by one-way ANOVA for each genotype to determine if flicker leads to significant molecular and cellular changes after stress. Statistical significance will be determined by a main effect (F-score) of p<0.05. Analysis of microglia depletion effects on behavioral and synaptic marker expression is performed by one-way ANOVA for each genotype to determine if microglia depletion alters flicker effects. Statistical significance is determined by a main effect (F-score) of p<0.05.

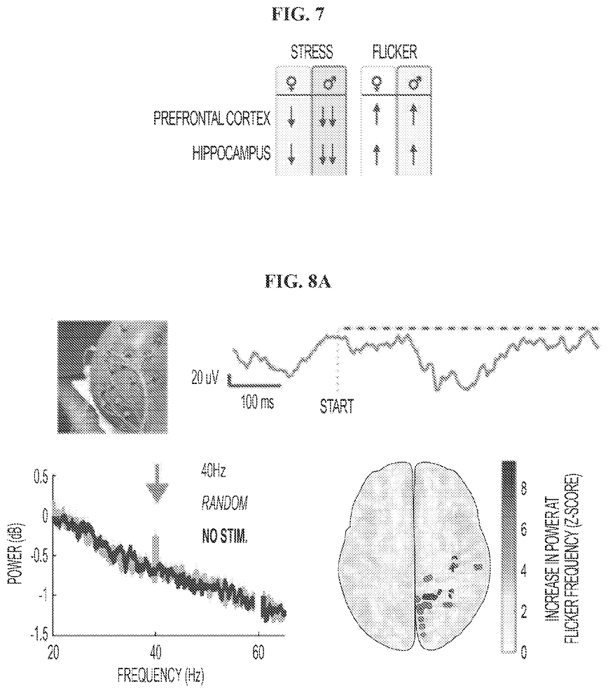

Results. Brain stimulation via audiovisual flicker reverses stress-induced molecular changes in synaptic marker expression induced by stress. Specifically, stress reduces synaptic marker expression in prefrontal cortex and hippocampus and flicker abolishes this stress-induced reduction in both WT, 5XFAD, and apoE4-KI mice in males and females (FIG. 7). Furthermore, the stimulation frequency that best promotes behavioral resilience in each sex (10 Hz for males and 40 Hz for female—see FIG. 2A) have the strongest effects on synaptic marker expression. Synaptic changes correlate with reduced proinflammatory, and phagocytosis related proteins. To identify transcriptomic and proteomic changes related to stress-resilience specifically, stress-resilient are compared to stress-susceptible mice across all genotypes and some pathways activated in resilient animals are consistent across genotypes. Second, the behavioral and synaptic effects of flicker following chronic stress are abolished with microglia depletion when using 40 Hz flicker to induce resilience (as in WT females) showing that these mechanism by which flicker acts depends on frequency and sex. The effects of other frequencies of stimulation, like 10 Hz which induces resilience in males, protect against synaptic loss but are not microglia dependent (FIG. 6). This is consistent with preliminary data and prior work showing that 40 Hz flicker transforms microglia while 20 Hz and 80 Hz flicker do not.²⁷ Third, 40 Hz flicker intervention protect against stress-induced microglia synaptic pruning as measured by synaptic engulfment in Thy1 female mice while 10 Hz flicker stimulation in males protects against stress-induced synaptic pruning in a microglia independent manner. This demonstrates for the first time how brain stimulation mitigates synaptic loss and pathological microglia-mediated synaptic pruning in a frequency-dependent manner. Furthermore, the results indicate the dependence of these effects on the presence of microglia.

### Example 5—Non-Invasive Targeting of Stress-Susceptible Memory and Emotion Circuits

Stress-susceptible memory and emotion circuits are not reachable by most existing non-invasive neurostimulation technology.^{21′-24′} Without intending to be bound by any particular theory of operation, it is believed that sensory flicker entrains local field potential (LFP) and modulates firing of single neurons in humans at a frequency matching that of sensory flicker. These responses in HPC, PFC, and AMY have been examined because these are stress-susceptible and key to the development of neuropsychiatric and cognitive symptoms.^{23,24′} Furthermore, optimal parameters of stimulation to modulate brain activity are determined, such as by varying frequency and sensory modality of the stimulation.

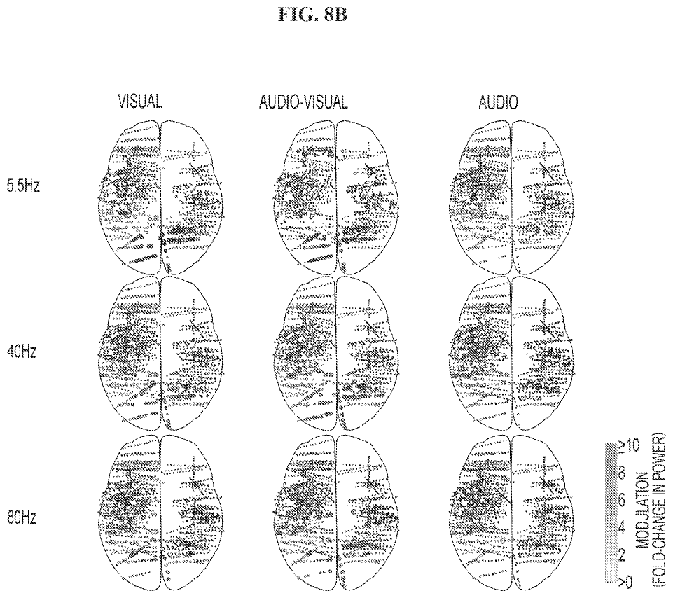

The intracranial neurophysiological effects of flicker in eleven participants were examined, which provide evidence in support of the underlying hypothesis (FIG. 8). Participants were exposed to 10 sec trials of 5.5 Hz, 20 Hz, 40 Hz, 80 Hz, or randomized audio, visual or audio-visual flicker while LFP was recorded in up to 150 different intracranial locations within single patients. In one participant, hippocampal single neuron and multi-neuron spiking activity was also recorded. As expected, LFP entrainment was found in sensory brain regions. Entrainment in HPC recording sites was also found (FIG. 8A, bottom-left). Finally, modulation of some single neurons was observed.

Accordingly, in FIG. 8A, top left, LFPs and single neurons were recorded in humans undergoing intracranial monitoring, while exposing participants to auditory flicker, visual flicker, or both at multiple frequencies. At the top right of FIG. 8, provided is one exemplary LFP trace (blue line) in HPC during 40 Hz audiovisual flicker shown from before and after the start of sensory stimulation (yellow and black bars respectively indicate when sound and light are on or off). As shown at the bottom left of FIG. 8A, LFP entrainment was found in hippocampal recordings as indicated by the peak in the power spectral density at 40 Hz during 40 Hz audiovisual flicker. The bottom right of FIG. 8A shows LFP entrainment across multiple circuits in four participants. Warmer colors indicate larger increases in power at the flicker frequency during stimulation, relative to no stimulation. FIG. 8B shows LFP entrainment across multiple circuits in eleven participants across multiple frequencies, including 5.5 Hz, 40 Hz, 80 Hz and multiple modalities, including audio, visual, and audio and visual together.

These preliminary results indicate that sensory flicker entrains stress-susceptible memory and emotion circuits in humans.

Furthermore, in a small feasibility trial in AD patients, it was found that all screened and enrolled participants entrained to 40 Hz audiovisual flicker in cortical regions detected via scalp EEG (He et al., under review). Participants experienced no severe adverse events related to flicker and some mild adverse events were possibly associated with treatment like dizziness and headache. All participants tolerated stimulation and adhered well to flicker therapy over 4-8 weeks with adherence rates of 95.5% on average. Finally, preliminary evidence was obtained that 8 weeks of gamma flicker altered cytokine levels and other immune factors in the cerebral spinal fluid. Thus, such experiments have established that chronic sensory flicker is safe and feasible, entrains cortical regions, and may affect neuroimmune signals in humans.

### Example 6—Additional Investigation Concerning Chronic Audio-Visual Flicker Exposure Protects Synaptic Marker Expression in Corticolimbic Brain Regions Following Chronic Stress in 5XFAD Mice and Flicker Effect on Excessive Synapse Remodeling and Neuropsychiatric-Like Behaviors by Suppressing the Complement Cascade

Chronic stress promotes excessive synaptic pruning via microglia, the primary immune cells of the brain. The resulting synaptic loss in corticolimbic regions leads to a 2-fold or more increased risk for neurodegenerative disease, such as Alzheimer's disease (AD). Driving specific frequencies of neural activity recruits immune cells and signals and on chronic stress, neuropsychiatric symptoms, and disease prevention, as described herein. Well timed flickering lights and sounds at predetermined frequencies, termed “flicker,” rapidly modulates immune signals in the brain with different effects based on the frequency of stimulation, presenting a powerful new tool to target neuroimmune dysfunction. This stimulation may be used reduce AD pathology and memory impairment in AD model mice. Also, according to principles described herein, chronic sensory flicker prevents neuropsychiatric-like behaviors in response to stress in a frequency dependent manner.

As disclosed herein, flicker stimulation prevents stress-induced synaptic loss and immune dysfunction. Individuals are twice as likely to develop disease, e.g., AD or other disease, following chronic or severe stress. The present disclosure provides new ways to prevent the development of such disease. Identifying the molecular mechanisms by which stimulation alters synapse density and neuropsychiatric-like behaviors provides insights into how neural activity affects the biology of stress and disease/AD risk.

The severe psychological and psychosocial stress experienced globally from the new coronavirus (COVID19) is expected to have lasting effects including significantly increasing stress-related disease globally. For example, individuals that have suffered from chronic stress or trauma have a 2-fold or greater increased risk of developing AD and experiencing AD-associated neuropsychiatric symptoms. Therefore, there is an unmet need for therapies that prevent or reduce the pathological effects of stress on neuropsychiatric and cognitive health. Such can result in a reduction in the prevalence of, e.g., AD. Loss of synaptic integrity is one of the best predictors of neuropsychiatric and cognitive decline. Chronic stress is thought to increase risk for neuropsychiatric symptoms by inducing synapse loss and dysfunction through the dysregulation of inflammatory signaling. In the healthy brain, synaptic pruning is initiated by the classic complement pathway which triggers microglia to selectively eliminate underused neuronal synapses while appropriate connections strengthen and mature. However under severe stress and other pathological states, classic complement activation is altered, leading to overactive microglia, which in turn promotes excessive and non-selective synaptic pruning in stress sensitive brain regions such as the prefrontal cortex (PFC) and hippocampus (HPC) (FIG. 1).

The resulting loss of synaptic integrity is thought to be the driving factor of changes in mood, neuropsychiatric health, and cognitive performance observed during prodromal stages of AD as well as during the progression of the disease. Indeed, complement signaling causes early synaptic loss in mouse models of AD while complement suppression protects against such loss.

Non-invasive sensory flicker stimulation has been shown to modulate inflammatory signaling and restore proper function of microglia in models of AD in a frequency-specific manner. Importantly, our preliminary data shows that sensory flicker stimulation boost resilience to stress-induced neuropsychiatric-like behaviors in male and female stressed mice (FIG. 2A). Together, these studies suggest that flicker treatment is a novel therapeutic intervention to restore adaptive microglia responses and synaptic health following chronic stress, reducing susceptibility to AD, other stress-driven diseases, and related neuropsychiatric symptoms (FIG. 1).

The present example is intended to show how sensory flicker stimulation mitigates synaptic loss following chronic stress in a model of AD pathology and the role of complement in these effects. These studies combine stress-induced microglia dysfunction in stress models and brain stimulation to manipulate brain immune function.

As reflected in FIG. 2A, adult male mice underwent either no stress and no flicker (control, grey), chronic unpredictable stress (CUS) alone (red), or CUS and 1 hr of flicker at 10 Hz (blue), 20 Hz (turquoise), or 40 Hz (green) for 1 hr/day for 30 days. From day 21 to 30, mice undergo open field, sucrose preference, novel object recognition, elevated plus maze, and forced swim tests. Results from each behavior are compared to no stress controls and combined into a composite stress susceptibility z-score that indicates more stressed (negative) or more resilient (positive) phenotypes. Left: Females that underwent CUS and 40 Hz flicker (green) exhibited less stressed behaviors than CUS alone (red) and were more similar to no stress control mice (grey). Right: Males that underwent CUS and 10 Hz (blue) or 20 Hz (turquoise) flicker behaved similarly to no stress control mice (grey). n.s. indicates not significant.

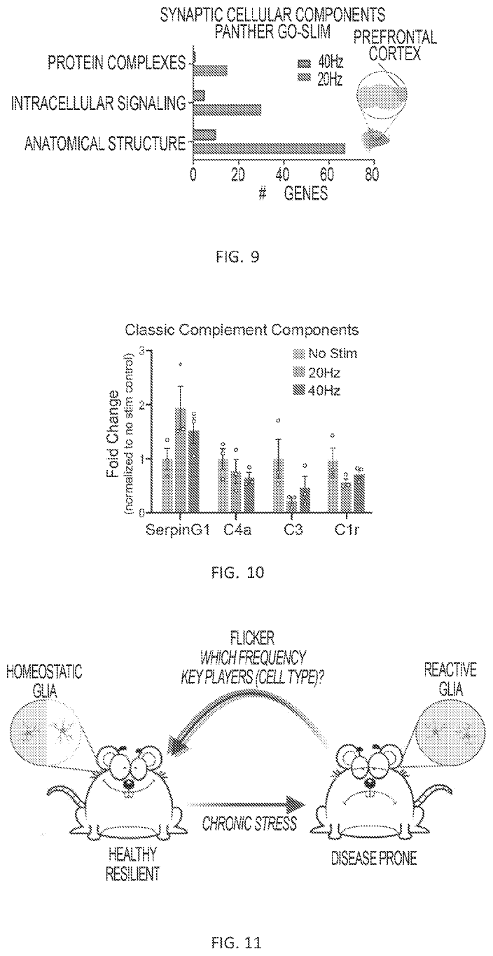

As reflected FIG. 9, audio-visual flicker induces up-regulation of synaptic genes. FIG. 9 illustrates that prolonged (1 hr/day for 21 days) 20 Hz (green) and 40 Hz (purple) audio-visual Flicker leads to significant upregulation of genes associated with complex formation, intracellular signaling and anatomical structure of synapses in prefrontal cortex with much more pronounced effects after 20 Hz Flicker. N=3 mice/group, 3 technical replicates. The transcriptomic results of FIG. 9 demonstrate support for the hypothesis and the feasibility of quantifying synaptic changes with high-throughput studies.

Preliminary studies show flicker stimulation enhances resilience and synaptogenesis. Clinical and preclinical evidence show that stress promotes neuropsychiatric and cognitive decline in AD by (1) decreasing dendritic arborization and spine density in corticolimbic brain regions, and (2) promoting the overproduction of proinflammatory cytokines leading to disruption of synaptic health and plasticity. As shown in FIG. 10, preliminary data shows that sensory flicker stimulation leads to a reduction in stress-induced neuropsychiatric-like behavior. Different frequencies of stimulation are optimal in male and female animals, suggesting that stimulation should be tuned to the sex of the subject. Preliminary transcriptomic data in FIG. 9 shows that chronic audio-visual flicker increases synaptogenesis signaling pathways in a frequency-dependent manner. Increased expression of synaptic markers in the PFC of wildtype (WT) male mice after chronic (1 hr/day for 21 days) audio-visual flicker exposure at 20 Hz with much smaller effects at 40 Hz. Furthermore, chronic flicker upregulates expression of Serping1 (C1NH), the primary inhibitor of the classic complement initiator C1q complex, while downregulating expression of downstream complement effectors (FIG. 10). Preliminary studies provide strong support for the hypothesis that flicker stimulation prevents stress-induced synaptic loss via suppression of the complement cascade. FIG. 10 illustrates audio-visual flicker suppresses classic complement signaling. Prolonged (1 hr/day for 21 days) 20 Hz (green) and 40 Hz (purple) flicker leads to robust upregulation of classic complement inhibitor SerpinG1 and downregulation of complement component expression compared to no stimulation (grey) in PFC. N=3 mice/group, 3 technical replicates.

Therefore, such non-invasive stimulation provides a novel therapeutic approach to prevent synaptic loss and reduce the risk of developing neurodegenerative disease, such as AD, which can be used prophylactically.

The use of multi-sensory flicker intervention prophylactically to treat stress pathology is novel. Preliminary data provides a compelling rationale for the use of audio-visual flicker for effective synaptogenesis in the context of stress. Second, using neurostimulation to prevent AD by resolving a major risk factor instead of treating after symptom onsite is original. Third, sex-specific brain stimulation to optimize responses for males and females has not been proposed before.

These studies open broad potential used for flicker at multiple frequencies beyond gamma in a wide range of contexts. Flicker is especially useful for studying neuroimmune interactions because it does not cause confounding immune effects due to invasive procedures. Furthermore, flicker effects were studied in non-stressed mice from a novel complement transgenic line which spontaneously develops neuropsychiatric-like behavior in young adult mice. Therefore, these tests illustrate how flicker effects classic complement, a key signaling cascade that regulates microglia, synaptic loss in AD and neuropsychiatric-like behaviors, revealing new roles for specific frequencies of neural activity in brain immune function.

### Research Design and Methods

Chronic audio-visual flicker exposure protects synaptic marker expression in corticolimbic brain regions following chronic stress in a mouse model of amyloidosis. Extensive evidence shows that chronic stress leads to functional and structural synaptic changes in HPC and PFC in a sexual dimorphic manner,⁴⁰⁻⁴² leading to sex-specific symptoms in stress-related diseases and AD. However, the use of noninvasive non-pharmacological sensory flicker intervention to protect against stress-induced synaptic pathology has never been studied. Preliminary data demonstrates that flicker promotes functional and structural gene expression changes at the synapse in a frequency-dependent manner (FIG. 9). The present premise is that flicker intervention before or during chronic stress exposure protects against sex-specific synaptic molecular changes in a frequency-dependent manner to prevent stress-induced synaptic loss. This premise may be tested by assessing synaptic marker expression in the 5XFAD mouse model of amyloidosis and WT littermates at 3 months of age in the pre-plaque stages before clear behavior deficits manifest because we are developing a preventative intervention. Synaptic deficits and loss in 5XFAD mice have been established, while relatively aggressive, 5XFAD mice have similar proteomic signatures to humans with symptomatic AD.

Experimental design: Male and female adult 5XFAD mice and WT littermates were be subjected to chronic unpredictable stress (CUS) for 21 days to induce pathological synaptic changes (FIG. 10). The CUS model is used to mimic the human experience of unpredictable stress in everyday life by using a combination of physical, psychological, and psychosocial stressors, resulting in molecular, cellular, structural, and functional changes associated with neuropsychiatric symptoms and neurodegenerative disorders including AD. This study includes four groups: (1) no stress/no flicker control, (2) CUS only, (3) CUS+10 Hz 1 hr/day, (4) CUS+40 Hz 1 hr/day in which animals are exposed to stressor and flicker daily but at different times. Frequencies effective for stress resilience have been determined to be different for males and females, for example, 10 Hz for males and 40 Hz for females. At the end of the 21 days, mice were sacrificed 1 hour after the last flicker treatment and PFC and HPC collected for Nanostring transcriptomic analysis (right hemisphere) and liquid chromatography with tandem mass spectrometry (LC MS/MS) analysis (left hemisphere) for an integrative within subject study (N=10/group). The number of animals required for each experiment was determined using power analysis for differences determined from preliminary studies (power=0.80 and alpha=0.05) and from published studies (N=10 per group per sex). For Nanostring: RNA samples will be prepped and sequenced on the Alzheimer's disease Nanostring panel (760 clinically AD implicated genes; panel includes neuronal and inflammatory markers) at Emory University's Integrated Genomics Core. For LC MS/MS: sample preparations and analysis will be performed at the Emory Integrated Proteomics Core. Synapse related markers associated with neuropsychiatric disease and AD including synaptosome associated protein 25 (SNAP25), complexin-2 (CPLX2), neurexin 3-alpha (NRXN3) and others, inflammatory genes involved in microglia activity (ex. TREM2; SLC2A5) and complement cascade activation (ex. Serping1; C1qa) were quantified. Flicker and stress effects were identified in and compared to transcriptomic and proteomic modules associated with cognitive decline and resilience in individuals with symptomatic or prodromal AD.

Analysis: Variance of gene expression and protein expression was performed separately using limma package of Bioconductor in R to identify differentially expressed genes (DEGs) and differentially expressed proteins (DEPs). Global gene ontology (GO) assessment was performed for DEGs and DEPs across brain regions in each sex separately. Rank-rank hypergeometric overlap (RRHO) test was performed to evaluate the degree of overlap in gene and protein signatures across brain regions in each sex separately. RRHO will be performed between sexes for each brain region. A comprehensive, unbiased analysis was generated across synaptic markers and immune factors, using partial least-squares discriminant analysis (PLSDA), as we have done before for cytokines. This approach generates a latent variable of combined measures that best separates stressed from unstressed groups and determines how stimulation shifts stressed animals along this axis. A determination if groups significantly differ in their scores along this latent variable using a one-way ANOVA with a main effect (F-score) of p<0.05 can be made and individual genes assessed using one-way ANOVAs corrected for multiple comparisons.

This study demonstrates how flicker prevents stress-induced loss of AD-relevant synaptic markers in HPC and PFC in each sex. There is upregulation of PFC and HPC synaptic marker expression in flickered and stressed mice compared to stressed mice without stimulation, with more protection following 40 Hz flicker in females and 10 Hz flicker in males. These changes in synaptic marker expression coincide with downregulation in complement component expression and upregulation of Serping1, which suppresses the complement cascade. as can be observed in FIG. 10. Finally, this allows for pro-inflammatory genes and markers of microglia phagocytosis to be suppressed following chronic audio-visual flicker as a function of suppressed complement cascade activation. This shows how chronic multi-sensory flicker protects synapse integrity from chronic stress in prodromal disease stages and this protection coincides with suppression of the complement cascade.

### Example 7 Sensory Flicker Intervention to Halt Stress-Induced Disease

Stress is thought to increase dementia risk by 2-fold or greater in part through acceleration of biological aging due to immune deregulation or “inflamm-aging”. In addition, the prevalence of stress-linked immune deregulation and neurodegeneration differs between males and females suggesting that the mechanisms involved in the etiology of stress-related neurodegeneration may be different between sexes. An urgent unmet need exists for therapies that prevent the pathological effects of stress to reduce the incidence of AD and address sex-specific risk of neurodegeneration. Our lab and collaborators have recently shown that non-invasive gamma frequency sensory flicker modulates inflammatory signaling and restores proper microglia function, the brain's innate immune cells, in a frequency-specific manner. Importantly, flicker interventions have improved cognitive function in animal models of AD. However, it remains unclear if flicker halts or reverses stress-induced inflamm-aging to reduce risk for AD due to stress. FIG. 11 illustrates a model to show sensory flicker methods boost resilience to stress-induced pathology and behavior deficits in a mouse model of neurodegenerative disease such as AD.

To investigate the efficacy of flicker mediated stress resilience in prodromal stages of AD, a well-established chronic unpredictable stress model was used to mimic the human experience of unpredictable stress in everyday life by using a combination of physical, psychological, and psychosocial stressors, resulting in molecular, cellular, structural, and functional changes associated with neuropsychiatric symptoms and neurodegenerative disorders including AD. The studies were conducted in wildtype C57BL6 adult mice and the 5XFAD amyloidosis mouse model to determine if chronic audio-visual flicker exposure can restore adaptive microglia profile in corticolimbic brain regions following chronic stress in the 5XFAD mouse model of AD. Functional changes in inflammatory markers expression (Olink inflammatory panel detection) and microglia morphology (3D reconstruction) will be quantified from four brain regions affected in AD of male and female stressed mice and To determine if molecular and cellular changes observed in Aim1 correlate to behavioral performance in a variety of tests used to assess cognitive and neuropsychiatric-like integrative within subject analysis. We hypothesize that sensory flicker will boost resilience to stress-induced inflamm-aging by modulating key neuroimmune regulators to promote behavioral improvement in a sex- and frequency specific manner.

Accordingly, innate immune signaling in the CNS under stress and flicker intervention can be characterized to profile disease vulnerable and resilience states. Restoration of beneficial microglial function following sensory stimulation flicker is thought to play a key role in flicker mediated cognitive improvement. For this reason, microglia morphology under stress in disease models can be characterized. In addition to microglia screening, the role of other CNS immune cells such as astrocytes should be understood. Recent evidence suggests that immune competent astrocyte may also play a role in AD progression and astrocytic biomarkers can be detected in brain and plasma in early stages of the disease. Under stress conditions, astrocytes acquire immune properties and are major producers of neuroinflammation. Reactive astrocytes undergo morphological changes like microglia and these changes can be used as a proxy for astrocyte mediated neuroinflammation.

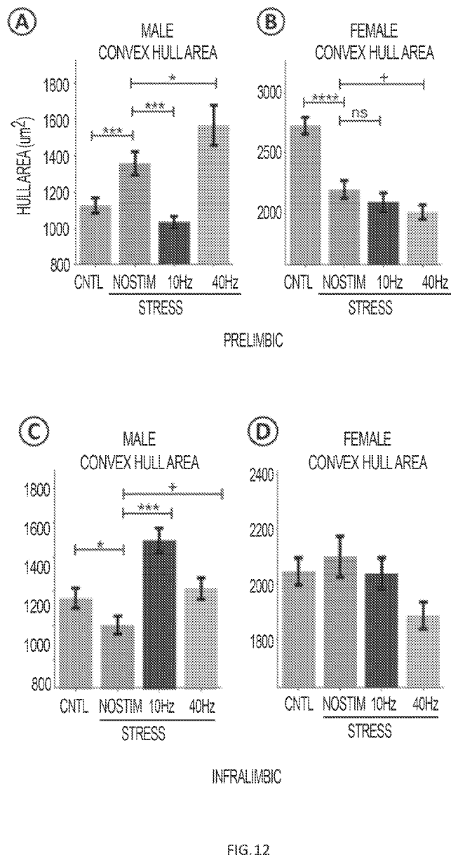

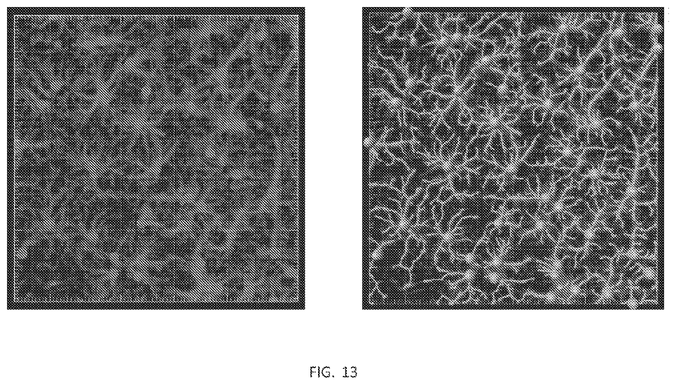

FIG. 12 illustrates how audiovisual flicker prevents stress induced astrocyte reactivity in a region-, sex- and frequency-specific manner. Astrocyte convex hull changes, a measure of volumetric cell spread, were quantified in (A) prelimbic male, (B) prelimbic female, (C) infralimbic male and (D) infralimbic female tissue. Audiovisual flicker prevents stress-induced astrocyte morphology changes in frequency previously shown to protect against stress induced behavioral deficits.

The present principles provide an analysis pipeline for glia (astrocyte and microglia) morphology profiling using wildtype C57BL6 stressed mice. Daily flicker intervention was introduced concomitantly with daily stress exposure (30 days) at multiple frequencies in male and female wildtype mice. The brains were harvested, acquired confocal images of GFAP labeled cells and conducted 3D reconstructions of astrocyte and microglia in the prefrontal cortex, an AD affected brain region highly susceptible to stress (FIG. 13). Using Imaris software for unbiased reconstructions of astrocytes, various morphology parameters including volumetric soma size, branch length, branch order, branching complexity, Sholl and convex hull analysis were quantified. Here it has been demonstrated that convex hull, a measure of total volumetric cell spread, is significantly altered by chronic stress exposure in cortical astrocytes. Most importantly, it has been shown for the first time that audiovisual flicker intervention can mitigate stress-induced astrocyte morphological changes in a regional-, sex- and frequency-specific manner (FIG. 13). This finding is meaningful as flicker mediated protection against astrocytic morphological changes coincides with behavioral improvement in wildtype male and female mice. Lastly, the functional outcome of microglia and astrocyte modulation under stress and flicker conditions can be investigated. Stress is thought to increase vulnerability to disease by promoting neuroinflammation and excessive synaptic pruning by microglia and astrocytes. To determine if morphological changes in microglia and astrocytes could lead to increased synaptic pruning, spine density in the prefrontal cortex and quantifying glia (microglia and astrocyte) interaction with spines were measured. Using super resolution confocal microscopy, microglia interaction with GFP-labeled spines on apical dendrites of the prefrontal cortex at was visualized at high resolution (FIG. 13). Optimization of this technique allows for reliably quantifying synaptic changes in stressed and flicker treated 5XFAD mice.

These results show for the first time that (1) chronic unpredictable stress induces neuropsychiatric-like behavioral deficits in 5XFAD male and female mice, (2) flicker promotes resilience to stress-induced cellular pathology in a frequency and sex-dependent manner. Super resolution imaging for glia-neuron interaction assessment can be captured. Glial changes in 5XFAD mice under stress and flicker and identify inflammatory biomarkers of stress resilience can be quantified. Identifying the effects of flicker on stress-induced glia dysregulation and behavior reveals a role for specific frequencies of activity in the development of AD-related symptoms and provide the foundation for using this non-invasive stimulation as a novel therapeutic approach to prevent cognitive and neuropsychiatric decline in AD.

### Example 8—Flicker Induced Modulation of Astrocytes Under Chronic Stress

72 male and female C57CL/6 Jackson mice were acquired for the flicker portion of this experiment. Mice were acclimated to the facility before beginning unpredictable stress exposure.

### CUS, Flicker, & Behavioral Testing

Animals were subjected to chronic unpredictable stress (CUS) exposure that consisted of a random combination of two stressors per day for 28 days. The stressors included wet bedding, lights on overnight, lights off during daytime, food and water deprivation overnight, 10% peppermint oil odor, radio static noise, 1 hr restraint, and 1 hr cage rotation. During CUS exposure, several groups were also treated with an audiovisual flicker to evaluate the effectiveness of several different frequencies at preventing the negative behavioral effects of stress. Mice administered CUS concurrently with Flicker therapy. There were four groups administered CUS, those receiving no flicker therapy, 10 Hz Flicker, 20 Hz Flicker, 40 Hz Flicker, and a control group that was not administered Flicker or CUS. These frequencies were selected based on previous evidence that gamma (40 Hz) and lower frequencies (≤20 Hz) modulate inflammatory signaling and affect glia reactivity in models of AD. Following Flicker exposure, an array of 7 behavioral tests were used to evaluate mood-associated and cognitive behavioral responses in the mice, including the sucrose consumption test, forced swim test, open field test, elevated plus maze, novel object recognition test, locomotor activity and social interaction test.

### Sectioning

Following behavioral analysis, mice were sacrificed and brains were extracted, then rinsed in 1× Phosphate buffered saline. Brains were fixed overnight in 4% paraformaldehyde in phosphate-buffered saline, then moved into a solution of 30% sucrose and left overnight. Brains were stored at −80° C. until sectioning.

Using a Cryostat (Leica CM1900), brains were sliced into 40 μm coronal sections. Sections containing regions of interest in the medial Prefrontal Cortex and dorsal Hippocampus (i.e., Prelimbic area, Infralimbic area, amygdala) were selected using the Allen Mouse Brain Atlas for reference. Sections were immediately placed in a cryoprotective solution of 30% glycerol, 30% ethylene glycol, and 10%. 2M Phosphate buffered saline and refrigerated at −20° C. until staining.

### Immunohistochemistry

5 animals of each sex were selected randomly from the control, CUS, 10 Hz, and 40 Hz groups. For each animal, tissue sections from the mPFC and dHPC were selected based on tissue integrity and damage. Following selection, sections were washed three times in 3 mL of wash solution, PBS with 1% donkey serum. Sections were then placed in 3 mL of blocking buffer (5% Donkey Serum PBS) for one hour. After blocking, sections were incubated in 1 mL of primary antibody solution of GFAP, the most common marker of astrocytes (1:1000 chicken GFAP in wash), overnight at 4° C. The next day, sections were washed three times in 3 mL of wash solution for ten minutes each. Samples were then incubated in the dark in 3 mL of secondary antibody solution (1:5000 anti-chicken 594) overnight at 4° C. On day three, sections were washed ten minutes each in 3 mL of wash solution. Sections were then stained with DAPI by incubating samples for 1 minute each in 2 mL of 1:500 DAPI in PBS. After a final wash for 2 minutes in PBS, sections were positioned in a bath of PBS, then mounted onto microscopy slides using PBS. After partial drying, Vectasheild was applied to the sections, then coverslips were applied and slides were sealed. After drying, slides were stored in the dark at 4° C. until imaging.

### Imaging

Using a Laser Scanning Confocal Microscope (Zeiss 700B), confocal images of tissue sections were acquired using a 20× objective. Images were taken as Z-stacks with a 1 μm step size, and maximum intensity projections were collected. For each sample, the CA3 region (dHPC), Infralimbic Cortex (PFC), and Prelimbic Cortex (PFC) were imaged for both the left and right brain. These brain regions were selected because they are particularly vulnerable to stress pathology and neurodegeneration and are key regulators of mood and cognitive processing. Over 7,000 astrocytes were imaged, then reconstructed in non-biased software.

### Reconstruction

Images were evaluated for astrocyte morphology factors including soma number, branch points, branch length, arborization area, and convex hull area. Soma number measures the number of astrocytes. Branch number and length refer to the number of process intersections and the length of the processes emerging from the cell soma respectively. Arborization area measures the area of process ramification while convex hull area quantifies the smallest three-dimensional area that includes all cell processes. Analysis was conducted using IMARIS, a non-biased, semi-automated 3D reconstruction system (Bitplane).

### Data Collection & Initial Analysis

To determine if morphological changes coincided with behavioral changes following stress and flicker intervention, we assessed behavioral response in seven different behavioral tests: sucrose consumption test, forced swim test, open field test, elevated plus maze, novel object recognition test, locomotor activity, and social interaction test. A z-score was generated and normalized to the female or male controls from the experiment, then corrected for the direction of effect, based on a previous study. A stress susceptibility score was determined by averaging behavioral test z scores for each animal. A dividing point was set at zero, with positive scores indicating a measure of resilience and negative scores indicating a measure of susceptibility.

### Statistical Testing

For each parameter, including soma number, branch points, branch length, arborization area, and convex hull area, a one-way ANOVA was first conducted to determine if treatment was a significant factor.

Between group comparisons were then evaluated using follow-up Tukey Tests. P values less than 0.01 were considered significant.

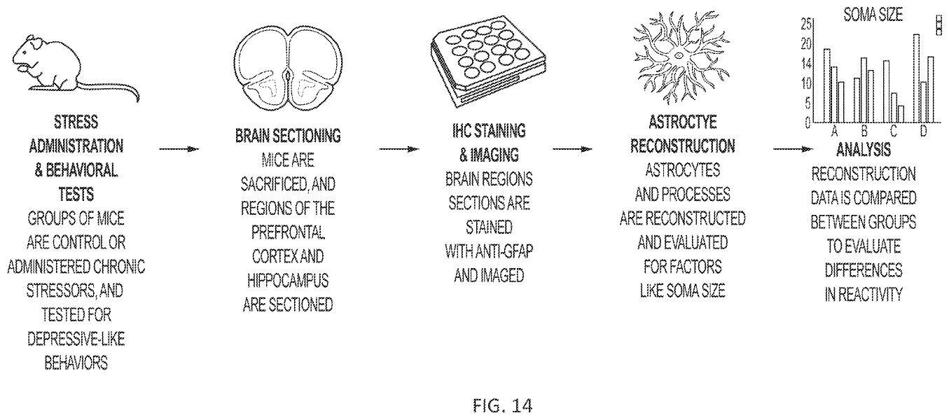

FIG. 14 illustrates a methods overview. Following stress administration and sacrifice, the cellular morphology of astrocytes was evaluated in mice.

### Results

Following the administration of CUS, region-specific, sex-specific, and frequency-specific differences in astrocyte morphology due to CUS and Flicker.

Flicker intervention promotes behavioral resilience in a frequency-specific manner.

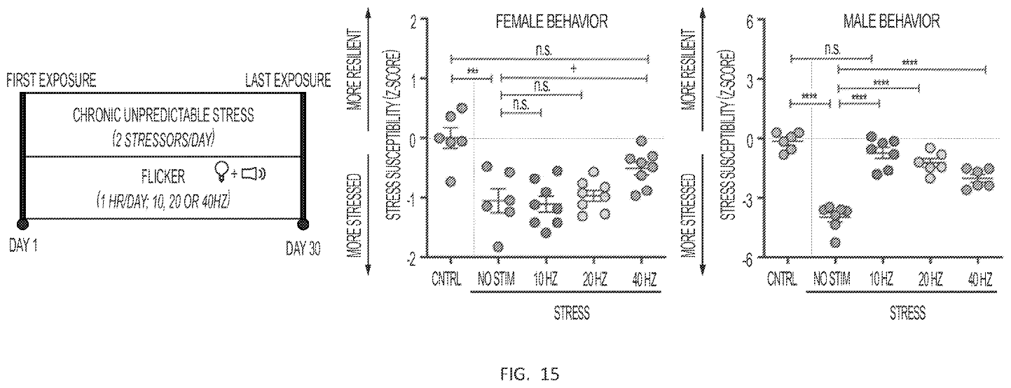

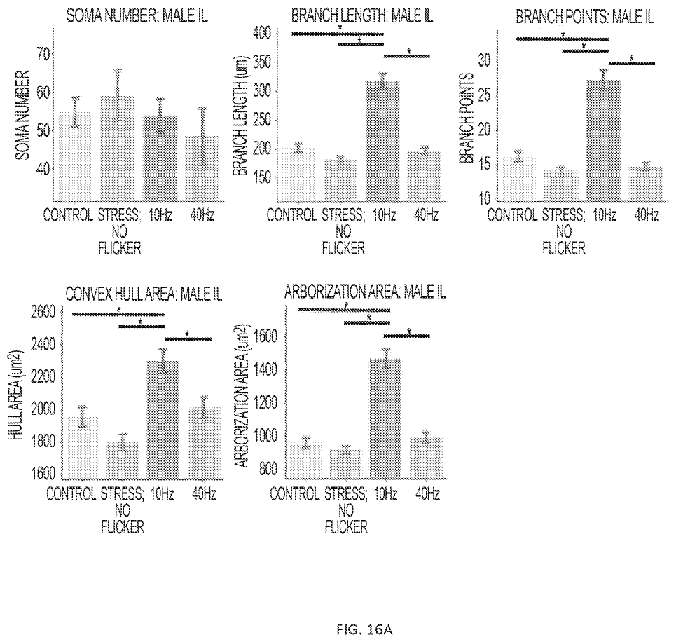

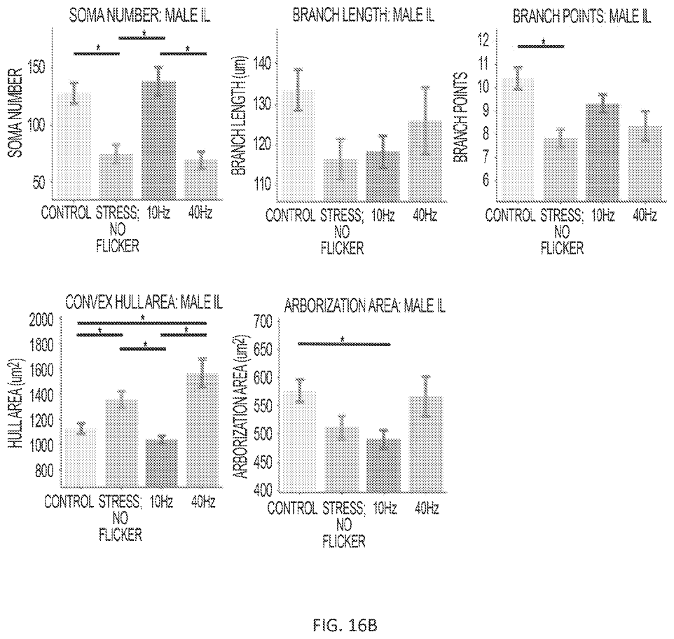

Compared to control mice, mice administered chronic unpredictable stress (CUS) without flicker intervention (FIG. 15; red) caused significant decreases in z-score. This indicates that chronic stress increases depressive behaviors in mice. Interestingly, males responded to chronic stress with a much greater deterioration in behavioral resilience as compared to females. Flicker administered during stress rescued behavior in mice at specific frequencies. Effective frequencies, however, varied by sex.

FIG. 15 illustrates results of a Flicker Intervention Study for Chronic Unpredictable Stress. Behavior Z-Scores were generated from 7 behavioral tests evaluating resilience and depression.

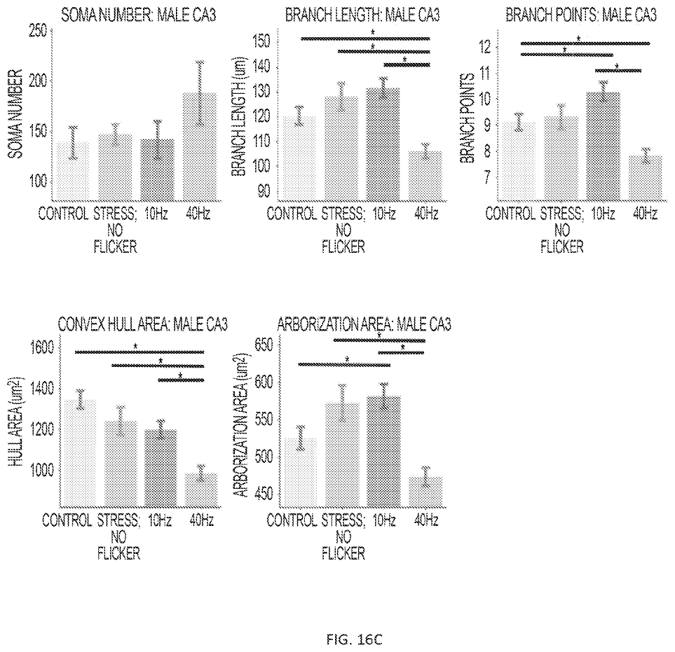

In this experiment, in male mice, 10 Hz Flicker was most effective for behavioral recovery. Males responded most to lower frequencies of Flicker (FIG. 16, dark blue). While significant improvements were made in behavior with each tested frequency, 10 Hz Flicker had the greatest effects, and was the only frequency with no significant differences in z-score compared to control. In this experiment in female mice, 40 Hz Flicker is most effective for behavioral recovery. Females responded most to higher frequencies of Flicker (FIG. 15, green). Lower frequencies of Flicker, 10 Hz and 20 Hz may not significantly improve behavior as compared to chronically stressed mice. 40 Hz flicker significantly improved behavior compared to the CUS group; further, there were no significant differences between behavior in the 40 Hz flicker group and control mice.

### Chronic Stress and Flicker Effects in Male Mice

Five parameters, soma number, convex hull, arborization area, branch length, and branch points, were evaluated to determine the number, spread, and complexity of astrocytes. Three regions were evaluated, the prelimbic cortex (PL) in the PFC, the Infralimbic cortex (IL) in the PFC, and the CA3 region in the Hippocampus. Regions evaluated are shown in FIG. 19.

### Male Infralimbic Region is Most Dramatically Affected by 10 Hz Flicker

FIG. 16A shows flicker intervention study in males. Astrocyte morphology in the Infralimbic region of the PFC. Graphs show results from follow-up Tukey Tests conducted after a one-way ANOVA. For Soma number, N=10,10,9,9 (control, CUS, 10 Hz, 40 Hz). For convex hull area, N=527,571,466,423. For branch points, branch length, and arborization area, N=567,593,495,439. * indicates significance with p<0.01.

As illustrated in FIG. 16A, within the Infralimbic cortex (IL), there were no significant changes in the number of astrocytes. Administration of CUS caused a non-significant decrease in spread of the astrocytes, shown by the decrease in convex hull area by on average 8%. Other measures of astrocyte morphology, including arborization area, branch length, and branch points, did not significantly change but did show a trend towards decreasing complexity following CUS. When Flicker was administered concurrently with CUS, astrocyte spread, measured by convex hull area, returned to baseline following 40 Hz Flicker, although changes were non-significant between control, CUS, and 40 Hz Flicker. 10 Hz Flicker increased convex hull area by an average of 17%, to well beyond baseline values. At 40 Hz, measures of complexity were slightly, and insignificantly, elevated compared to chronic stress values. At 10 Hz, branch points, branch length, and arborization area were significantly elevated past CUS and control values.

### Male Prelimbic Region Shows Regional Effects of Flicker

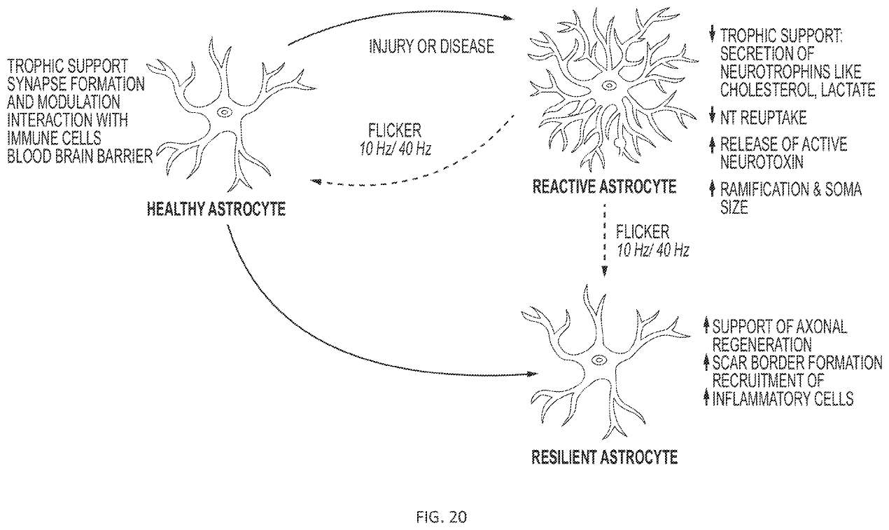

FIG. 16B shows flicker intervention study in males. Astrocyte morphology in the Prelimbic region of the PFC. Graphs show results from follow-up Tukey Tests conducted after a one-way ANOVA. For Soma number, N=4,5,6,2 (control, CUS, 10 Hz, 40 Hz). For convex hull area, N=499,360,811,141. For branch points, branch length, and arborization area, N=507,376,988,143. * indicates significance with p<0.01. As shown in FIG. 20B effects both varied in the PL compared to the IL.

Within the PL, astrocyte number was affected by CUS and Flicker administration. Following CUS, soma number decreased significantly. While 40 Hz Flicker did not affect soma number, 10 Hz Flicker returned soma number to the level of controls. Administration of CUS caused increasing astrocyte spread by an average of 21% compared to control. Branch points showed a significant decrease following CUS, while branch length and arborization area showed decreasing trends compared to controls.

When Flicker was administered with CUS, effects were highly variable depending on frequency. 10 Hz Flicker did not significantly affect arborization area or branch length compared to CUS values. 40 Hz Flicker, however, caused increasing trends in both measures, returning the values to baseline levels.

Another measure of complexity, branch points, only showed increasing trends compared to CUS following 10 Hz Flicker, not with 40 Hz Flicker. Spread of the cells, as indicated by convex hull area, decreased following 10 Hz Flicker by 8%. 40 Hz Flicker caused greater increases in area than CUS, increasing area by 40% compared to control.

### CA3 Region Indicates Differential Effects of Flicker on the Dorsal Hippocampus

FIG. 16C shows Flicker intervention study in Males. Astrocyte morphology in the CA3 region of the dHPC. Graphs show results from follow-up Tukey Tests conducted after a one-way ANOVA. For Soma number, N=6,4,6,2 (control, CUS, 10 Hz, 40 Hz). For convex hull area, N=782, 295, 800, 690. For branch points, branch length, and arborization area, N-899, 298, 822, 702. * indicates significance with p<0.01.

There were no significant changes in soma number in the CA3 region except for significant increases following 40 Hz Flicker (Figure V.C.). Administration of CUS decreased spread of astrocytes, with convex hull area decreasing by 8%. Complexity showed increasing trends in the CA3 region following CUS when considering arborization area and branch length. There was no change in the number of branch points in controls compared to CUS.

When Flicker was administered with CUS, 10 Hz groups showed increasing complexity, with significantly increased values of branch points and arborization area. 40 Hz groups showed significant changes in morphology by almost every measure. Spread of the cells measured by convex hull area decreased dramatically compared to all other groups, by 27%. Complexity decreased significantly by all measures. Branch length, branch points, and arborization area all showed significant decreases as compared to CUS, and 10 Hz Flicker, and decreases in branch length and arborization area compared to controls.

### Chronic Stress and Flicker Effects in Female Mice

### Female Infralimbic Region is Most Dramatically Affected by 40 Hz Flicker

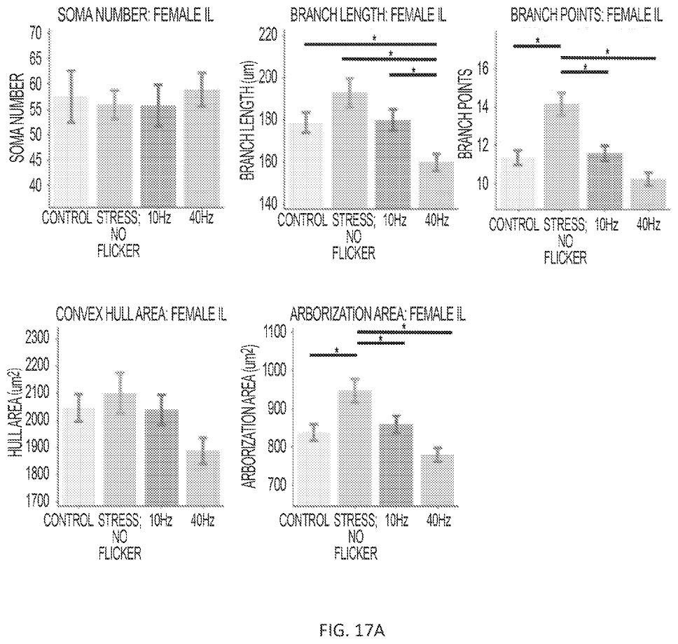

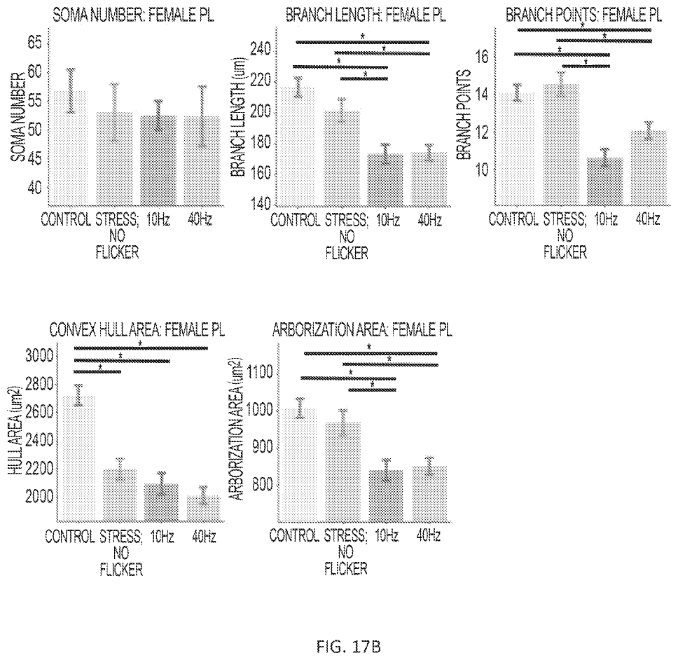

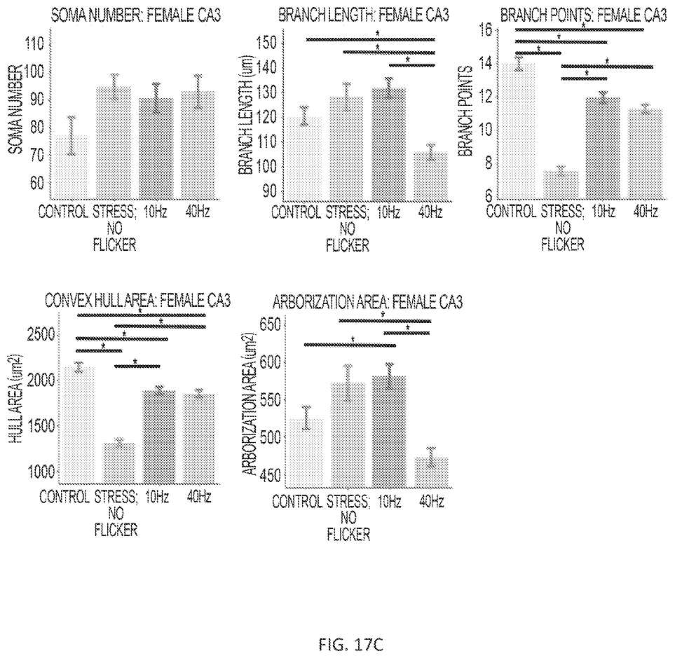

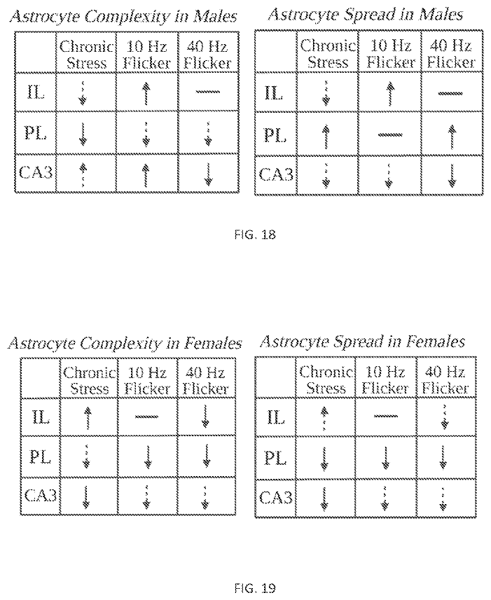

FIG. 17A shows Flicker intervention study in Females. Astrocyte morphology in the Infralimbic region of the PFC. Graphs show results from follow-up Tukey Tests conducted after a one-way ANOVA. For Soma number, N=10,7,10,8 (control, CUS, 10 Hz, 40 Hz). For convex hull area, N=542,379,531,451. For branch points, branch length, and arborization area, N=575,395,569,478. * indicates significance with p<0.01.

Compared to the male groups, female groups varied in their response to stress and flicker intervention, while showing similar variance in effect based on region. Within the Infralimbic cortex (IL), there were no significant changes in the number of astrocytes. Administration of CUS caused significant increases in astrocyte complexity and increasing trends in spread. This was showcased by significant increases in arborization area and branch points, and only increasing trends in convex hull area and branch length.

Both frequencies of Flicker produced significant changes in astrocyte morphology in the IL region. Both frequencies decreased measures of spread and complexity compared to chronic stress groups, and the 40 Hz Flicker was the most impactful. In all measures of spread and complexity, 10 Hz Flicker prevented significant changes in morphology compared to control groups. 40 Hz Flicker, while trending in the same direction as control, produced significant decreases greater than control or 10 Hz Flicker group levels in branch length, arborization area, branch points, and convex hull area.

Flicker does not have Frequency-Specific Effects in the Prelimbic Region

FIG. 17B shows Flicker intervention study in Females. Astrocyte morphology in the Prelimbic region of the PFC. Graphs show results from follow-up Tukey Tests conducted after a one-way ANOVA. For Soma number, N=9,6,8,8 (control, CUS, 10 Hz, 40 Hz). For convex hull area, N=480,306,396,402. For branch points, branch length, and arborization area, N=513,322,429,423. * indicates significance with p<0.01. As shown in FIG. 17B, within the Prelimbic cortex (PL), there were no significant changes in the number of astrocytes.

CUS produced dramatic, significant changes in spread of cells, shown by decrease in convex hull area by 19%. There were no changes in branch points, while arboirizaiton area and branch length showed decreasing trends in CUS groups.

Flicker administration did not significantly change convex hull area values from CUS groups, though Flicker did continue to trend cell spread smaller, with the smallest area being the 40 Hz group decreasing by 26%. All other measures, including branch length, branch points, and arborization area, significantly decreased compared to control and CUS groups, and there were no significant changes between 10 Hz and 40 Hz Flicker.

### Flicker is Protective Against CUS in the CA3 Region in a Non-Frequency-Specific Manner

FIG. 17C shows flicker intervention study for chronic unpredictable stress in females. Astrocyte morphology in the CA3 region of the dHPC. Graphs show results from follow-up Tukey Tests conducted after a one-way ANOVA. For Soma number, N=8,6,8,8 (control, CUS, 10 Hz, 40 Hz). For convex hull area, N=581,462,689,676. For branch points, branch length, and arborization area, N=620,573,726,753. * indicates significance with p<0.01.

There were no significant changes in soma number, although there was an increasing trend in all groups compared to controls (Figure VI.C). CUS produced dramatic and significant changes in spread and complexity throughout the CA3 region. All measures, including convex hull area, arborization area, branch length, and branch points, decreased significantly with CUS administration. Of note, convex hull area decreased by 39%.

Administration of Flicker did not prevent significant changes in morphology due to CUS but did decrease its effect. Convex hull area was increased compared to CUS groups, at 12% and 14% for 10 Hz and 40 Hz Flicker respectively. Other measures, including branch points, branch length, and arborization area, showed the same pattern, with a partial recovery when Flicker was administered, and no changes between different frequencies of Flicker.

### Discussion

Chronic stress has been shown to worsen symptoms of neurological and neuropsychiatric disease and can increase occurrence of neurological and neuropsychiatric conditions. Previous studies have associated chronic stress with changes in neuron functionality, showing that stress limits synaptic function and long-term potentiation.²³ Stress is also associated with astrocyte reactivity, and previous work has shown that astrocyte reactivity is associated with a collection of morphological changes in the cell, including hypertrophy of cell bodies, enlargement of processes, increased volume, and hyperramification. In this study, we first determined that Flicker could prevent negative changes in behavior due to stress in frequency-specific manner that varied based on sex. We next established the effects of chronic unpredictable stress on astrocyte cell morphology.

Then, when looking at cellular morphology under flicker intervention, it can be seen that the effects of Flicker are dependent on sex, frequency, and region.

FIG. 18 shows a Male Morphology Summary. The tables show changes in morphology compared to controls, looking at complexity as measured by branch points and arborization area (left) and spread as measured by convex hull area (right). Dotted arrows indicate non-significant trends, and undotted arrows indicate significant changes. Straight lines refer to no change compared to controls.

### Male Morphology Recovery

While CUS causes increasing spread in PL male astrocytes, it also decreases complexity in cells. Overall, in the PL CUS increased astrocyte spread (CH) and 10 Hz flicker returned this measure to baseline while 40 Hz Flicker further increased spread. Complexity decreased following CUS. While 10 Hz Flicker was not protective against morphological changes in complexity in this region, 40 Hz Flicker prevented CUS-related complexity decreases. If PL astrocytes are playing a role in the behavioral effects observed in Figure III, this would indicate that the protective effects of Flicker are most relevant with regard to astrocyte spread, followed by branch point complexity changes, in the PL region.

In contrast, astrocytes in the male IL region decreased in complexity and spread following CUS; however, these changes were in large part insignificant. Therefore, while 10 Hz Flicker did produce significant changes in astrocyte morphology of this region, it is more likely that the PL region is modulating the effects of chronic stress and Flicker.

When looking at the effect of 40 Hz Flicker in the CA3 region for male mice, data seems to indicate that 40 Hz negatively affects astrocytes in the CA3 region of males. Perhaps, this data may indicate that the variant effects by region represent a protective effect in some areas, like 40 Hz Flicker administration for the male PL, while in other regions the effects of Flicker are damaging, as the CA3 region seems to indicate with 40 Hz related changes in astrocyte morphology. In males, there were significant benefits of 40 Hz Flicker for behavioral resilience; however, only 10 Hz Flicker was fully protective against CUS-related behavioral changes. This could indicate that in some regions, astrocyte morphology is more relevant to behavior. In males, IL region is the most likely candidate, due to recovery of astrocytes after chronic stress, especially at 10 Hz frequency Flicker.

However, another possibility is that the behavioral effects are a conglomerate representation of the positive and negative astrocyte changes in the brain. If 10 Hz Flicker was beneficial in the IL region and 40 Hz Flicker was beneficial in the PL region, the discrepancy in the effects of the frequency might be because 40 Hz Flicker changed astrocyte morphology in the Hippocampus (CA3 region).

19 shows Female Morphology Summary. Charts show changes in morphology compared to controls, looking at complexity as measured by branch points and arborization area (left) and spread as measured by convex hull area (right). Dotted arrows indicate non-significant trends, and undotted arrows indicate significant changes. Straight lines refer to no change compared to controls.

### Female Morphology Recovery

In comparison to the highly divergent response of male astrocytes to Flicker, female astrocytes showed less frequency-specific effects. In both the PL and CA3 regions, Flicker caused significant changes in astrocyte morphology that did not vary between 10 Hz and 40 Hz. Looking at the PL region, Flicker seemed to have a greater effect on astrocyte spread and complexity than CUS, producing significant decreases in both measures. Because this region was not impacted greater because of stress, and because Flicker trends were in the same direction as CUS trends, this indicates that the PL region is not likely driving behavioral resilience in females. In another region, CA3 of the Hippocampus, Flicker does seemingly protect astrocyte morphology in a non-frequency specific manner. Changes in this region due to CUS that include dramatic decreases in complexity and spread show significant improvement in both 10 Hz and 40 Hz Flicker groups.

In the IL region, while 10 Hz Flicker was able to prevent increases in spread and complexity due to CUS, 40 Hz Flicker surpassed control group levels. 40 Hz produced significant decreases in spread and complexity compared to controls, and each of these changes was in the opposite direction of changes due to chronic stress. Does this indicate that Flicker has effects on its own beyond preventing changes due to chronic stress? As these changes are in a behaviorally relevant region and are most associated with the beneficial female frequency, this could also indicate that these changes are protective against stress. That being, Flicker in the IL region may cause changes in astrocyte morphology beneficial even in the absence of stress. Further study needs to be conducted in this region to determine whether these changes are still present in the absence of CUS administration. This would determine whether Flicker alone induces changes in astrocyte reactivity.

### Effective Frequencies and Future Directions

This data seems to indicate that the male PL and the female IL are the most promising targets for Flicker intervention. These regions show the most recovery at effective flicker frequencies (10 Hz in males and 40 Hz in females). Further, when considering other regional effects, it also seems that the effects of Flicker may be additive. That is, rather than effective frequencies are the sum of the effect of that frequency in several regions of the Hippocampus and Prefrontal cortex. Whether this means that Flicker changes baseline astrocyte reactivity, is protective against chronic stress-related changes, or induces another astrocyte state remains to be determined. However, it is clear that the effects of stress and Flicker are complex. Our results indicate flicker may be mediating recovery of behavior by promoting resilient astrocyte function. Stress significantly changes astrocyte morphology, and Flicker can prevent these changes in a manner suggesting that astrocyte morphology correlates with behavioral changes. Further study is being conducted to determine whether the morphological changes observed in the astrocytes are associated with synaptic changes in excitatory neurons of the regions. If our theory is correct, astrocytes in specific regions will show a reduction in non-specific synapse engulfment following the administration of effective flicker frequencies.

Based on morphology data, cells in the hippocampal region may show the most recovery at flicker frequencies different from those associated with chronic stress recovery. In the CA3 region, males saw the greatest resilience in astrocyte morphology at 40 Hz while females saw the greatest resilience at 10 Hz. We expect that these frequencies may be the most effective for memory function. Similar to the IL region being associated with behavioral recovery, we expect that the hippocampal region might be similarly affected by the observed cellular changes.

Astrogliosis causes an immune reaction in the brain that can change neuronal health and function. Neurodegenerative diseases associated with inflammation and chronic stress are also highly associated with astrogliosis, a reactive state in astrocytes induced by injury or disease. In this study, we identify a potential non-invasive intervention to prevent astrocyte-mediated changes triggered by stress. Audiovisual Flicker has a clear effect on behavior, and our study has shown how the treatment affects the most abundant glial cells in the brain, the astrocyte. We first identified specific frequencies effective for behavioral recovery, then confirmed that these changes are associated with astrocyte recovery or resilience in brain regions most affected by chronic stress. Our study has potentially identified a feasible intervention for stress-related inflammation, which could be especially impactful in populations most vulnerable to inflammatory disorders.

FIG. 20 illustrates Astrocyte reactivity after Chronic Stress and Flicker. Healthy astrocytes maintain the blood barrier, support synapse formation, and provide trophic support to neurons. After injury or disease, astrocytes can become reactive, losing neuroprotective functions and promoting neuronal death. Audiovisual flicker is thought to prevent astrocyte reactivity or restore a resilient astrocyte phenotype.

As discussed above, Severe and/or chronic psychological and psychosocial stress increases life-long risk for stress-related conditions such as depression and generalized anxiety. Stress promotes neuropsychiatric decline in part by promoting the dysregulation of glia cells in stress sensitive brain regions such as the prefrontal cortex (PFC) leading to disruption of synaptic health and plasticity. As discussed above, it has been demonstrated that non-invasive gamma (40 Hz) sensory flicker stimulation modulates inflammatory signaling and restores proper function of microglia in models of Alzheimer's disease (AD). As described herein, sensory flicker stimulation can mitigate neuropsychiatric-like behavioral deficits and rescue glia pathology following chronic stress.

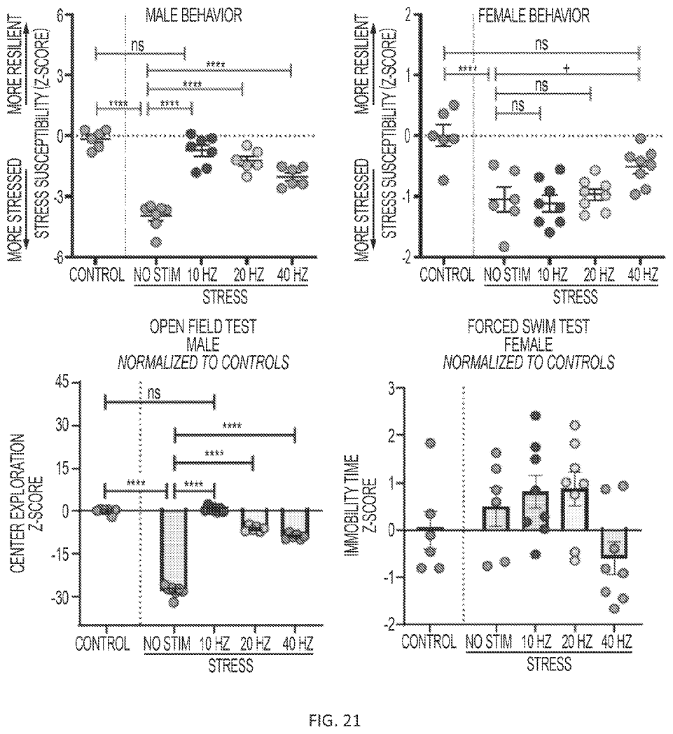

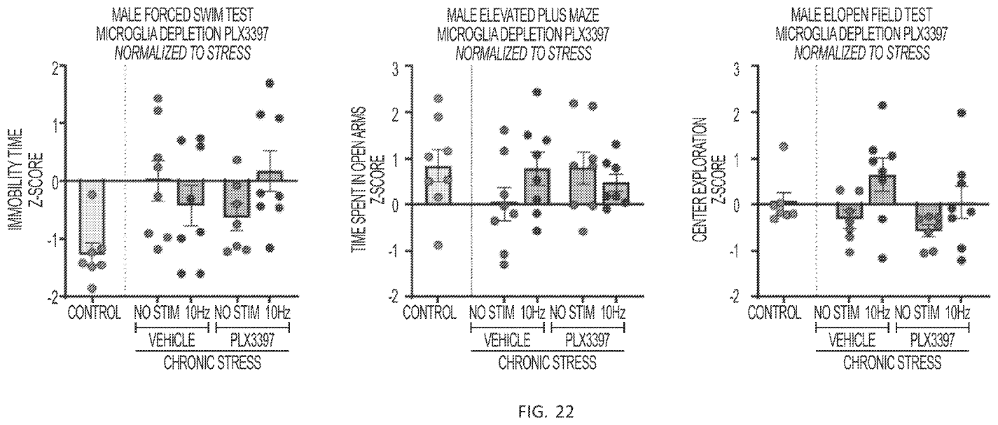

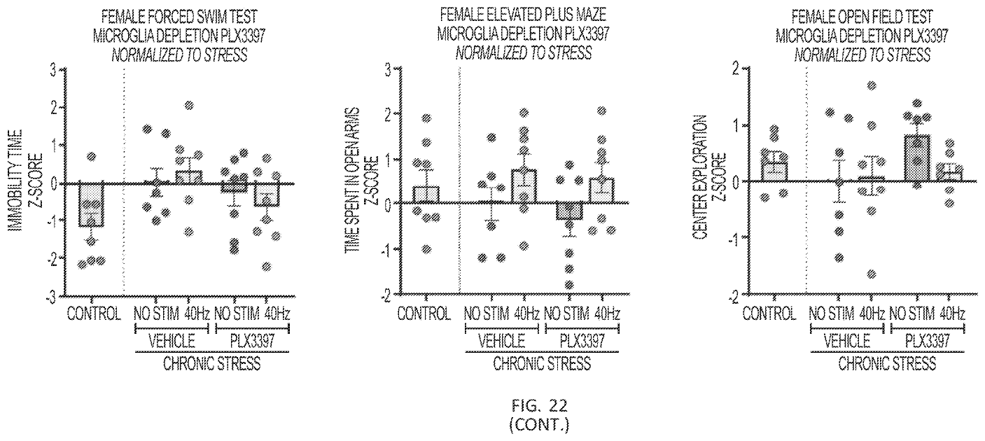

FIG. 21 further shows chronic audiovisual flicker boosts resilience to stress. The top panels show stress susceptibility under flicker intervention or stress alone conditions. The lower panels show stress susceptibility in the open field test and forced swim test under flicker intervention and stress alone. + indicates ρ˜0.05, ρ˜0.0001 FIG. 22 shows effects of microglia depletion on behavior and illustrates that microglia play a causal role in these behaviors and the effects of flicker in a sex-specific manner.

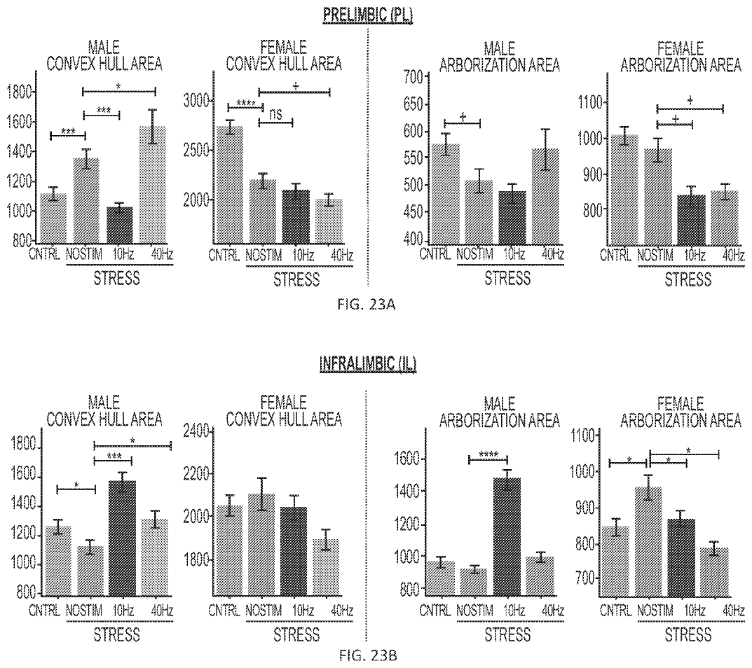

FIGS. 23A and 23B illustrate that stress leads to frequency-, sex- and region-specific morphological shifts. Morphological parameters are presented for the prelimbic (Right, orange) and infralimbic (left, purple) regions show regional differences in astrocyte morphological shift following stress+flicker or stress alone conditions in male and female mice.

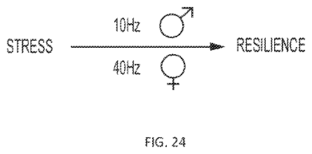

FIG. 24 illustrates sensory flicker intervention boosts resilience during maladaptive stress exposure. As described herein, the medial prefrontal cortex is sensitive to stress and sensory flicker modulation in males and females. Sensory stimulation flicker protects against stress-induced behavioral changes in a sex and frequency specific manner.

According to the principles described herein a method for inducing neurological response in a subject can include selecting a frequency for application of non-invasive brain stimulation to which a subject is to be exposed and exposing the subject to the non-invasive brain stimulation at the selected frequency in a range of about 5-100 Hz.

The frequency may be selected according to a gender of the subject.

In some circumstances, the subject is not currently subject to a diagnosis of a neurological or neuropsychiatric disorder, and wherein the subject is selected for exposure to the brain stimulation based on (i) the subject's previous exposure to a stressor, (ii) the subject's contemporaneous exposure to a stressor, or (iii) an anticipated exposure of the subject to a future stressor.

The exposure of the subject to non-invasive brain stimulation may be for the prophylactic treatment of stress or a neurogenerative disorder resulting from exposure to stress.

According to the principles described herein, the subject experiences increased resistance to stress-induced pathology.

The exposure of the subject to the non-invasive brain stimulation can occur at a fixed time on a daily basis.

The non-invasive brain stimulation can be audiovisual flicker.

The non-invasive brain stimulation can occur at a frequency of about 20 Hz.

The method can include exposing the subject to the non-invasive brain stimulation for about 15 to 90 minutes per day for seven or more days.

The method may further include assessing the efficacy of the non-invasive brain stimulation in the subject following the exposure of the subject to the sensory stimulation.

The assessment can include performing a stress assessment of the subject. The assessment can include a measurement of one or more biomarkers of stress-induced pathology in the subject. The biomarkers can include cytokines. The stress-induced pathology may be synaptic loss or neuronal atrophy. The stress-induced pathology may be immune dysregulation.

The method may include determining whether to modulate one or more parameters of the sensory stimulation based on the results of the assessment. The parameters may include frequency of the non-invasive brain stimulation, duration of the non-invasive brain stimulation per treatment episode, time of day of the non-invasive brain stimulation, or any combination thereof.

The method may further include exposing the subject to sensory brain in which the one or more of the parameters of the non-invasive brain stimulation have been modulated.

The non-invasive brain stimulation may reduce risk of or expression of anxiety, depression, aggression, anhedonia, decreased cognitive performance due to stress, or neurodegenerative disease in the subject. The non-invasive brain stimulation may increase synaptic marker expression. The non-invasive brain stimulation may alter microglia. The non-invasive brain stimulation may alter cytokine expression.

In some cases, the subject may not currently be subject to a diagnosis of a neurodegenerative disease.

The subject may be selected for treatment by exposure to the brain stimulation by assessing the subject's susceptibility to stress-induced pathology. The assessment of the subject's susceptibility to stress-induced pathology may include an anxiety assessment, an anhedonia assessment, a measurement of one or more biomarkers of stress pathology in the subject, or a combination thereof. The frequency of the brain stimulation may be selected based on the stress-induced pathology to which the subject is susceptible. The frequency of the brain stimulation is selected based on the identity of a region within the subject's brain for which brain stimulation is desired. The frequency of the brain stimulation may be selected for stimulation of the subject's hippocampus (HPC), amygdala (AMY), prefrontal cortex (PFC), nucleus accumbens (NAc), or any combination thereof.

The subject may be in remission from a neuropsychiatric disorder. The selected frequency for a female subject may be greater than the selected frequency for a male subject. The selected frequency may be in the range of about 10 hz to about 20 Hz for a male subject, for example, 10 Hz. The selected frequency may be in the range of about 30 to about 40 Hz for a female subject, for example, 40 Hz.

The subject may not currently be subject to a diagnosis of a neurological or neuropsychiatric disorder.

The subject may be selected for exposure to the brain stimulation based on (i) the subject's previous exposure to a stressor, (ii) the subject's contemporaneous exposure to a stressor, or (iii) an anticipated exposure of the subject to a future stressor

According to the principles described herein, a method for increasing resilience to stress-induced pathology in a subject may include exposing the subject to non-invasive brain stimulation that is effective to drive oscillations, rhythmic electrical activity, or both in the subject at a target frequency of about 5-100 Hz, wherein the target frequency is based on the subject's gender, wherein the subject is not currently subject to a diagnosis of a neurological or neuropsychiatric disorder, and wherein the subject has been assessed as having an elevated genetic risk for a neurological or neuropsychiatric disorder.

According to the principles described herein, a method for treating a neurological or neuropsychiatric disorder in a subject for whom an antidepressant or anti-anxiety medication is contraindicated or who is resistant to treatment with an antidepressant or anti-anxiety medication may include exposing the subject to brain stimulation that is effective to drive oscillations, rhythmic electrical activity, or both in the subject at a target frequency of about 5-100 Hz, wherein a target frequency is based on the subject's gender.

In any aspect described herein, the subject may pregnant or nursing.

According to the principles described herein, a method for treating dysregulation of classic complement signaling, dysregulation of microglia activity, neuroinflammation, or pathological synaptic change in a subject may comprise exposing the subject to brain stimulation that is effective to drive oscillations, rhythmic electrical activity, or both in the subject at a target frequency of about 5-100 Hz, wherein a target frequency is based on the subject's gender, wherein the subject is selected for exposure to the brain stimulation based on a determination of neuroinflammation, dysregulation of classic complement signaling, dysregulation of microglia activity, or pathological synaptic change in the subject, or, wherein the subject is selected for exposure to the brain stimulation based on a determination that the subject has an elevated genetic risk for a condition having a classic complement component.

The dysregulation of classic complement signaling may be characterized by elevated classic complement signaling relative to a reference value corresponding to a normal level of classic complement signaling.

The dysregulation of classic complement signaling may be characterized by decreased classic complement signaling relative to a reference value corresponding to a normal level of classic complement signaling.

The dysregulation of microglia activity may be characterized by elevated microglia activity relative to a reference value corresponding to a normal level of microglia activity.

The dysregulation of microglia activity may be characterized by decreased microglia activity relative to a reference value corresponding to a normal level of microglia activity.

The subject may be selected for exposure to the brain stimulation based on a determination that the subject has an elevated genetic risk for bipolar disorder, amyotrophic lateral sclerosis, Parkinson's disease, attention deficit-hyperactivity disorder, obsessive-compulsive disorder, multiple sclerosis, systemic lupus, autism, inflammatory bowel disease, type-2 diabetes, or age-related macular degeneration.

According to principles described herein, a method for increasing resilience to stress-induced pathology in a subject may include exposing the subject to brain stimulation that is effective to drive oscillations, rhythmic electrical activity, or both in the subject at a frequency of about 5-100 Hz, wherein the subject is not currently subject to a diagnosis of a neurological or neuropsychiatric disorder, and, wherein the subject is selected for exposure to the brain stimulation based on (i) the subject's previous exposure to a stressor, (ii) the subject's contemporaneous exposure to a stressor, or (iii) an anticipated exposure of the subject to a future stressor.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed:

1. A method for treating a neuropsychiatric or neurological disorder by inducing neurological response in a, the method comprising:
selecting a frequency for application of non-invasive brain stimulation to which a subject is to be exposed;
exposing the subject to the non-invasive sensory stimulation at the selected frequency in a range of about 5-100 Hz.

2. The method according to claim 1, wherein the frequency is selected according to a gender of the subject.

3. A method of applying non-invasive brain stimulation to a subject, the method, comprising:
selecting a frequency for application of non-invasive brain stimulation to which a subject is to be exposed, according to a gender of the subject;
exposing the subject to the non-invasive brain stimulation at the selected frequency in a range of about 5-100 Hz.

4. The method of any one of the preceding claims, wherein the non-invasive brain stimulation applied is sensory flicker.

5. The method of any one of the preceding claims, wherein the subject is not currently subject to a diagnosis of a neurological or neuropsychiatric disorder, and wherein the subject is selected for exposure to the brain stimulation based on (i) the subject's previous exposure to a stressor, (ii) the subject's contemporaneous exposure to a stressor, or (iii) an anticipated exposure of the subject to a future stressor.

6. The method of any one of the preceding claims, whereby the subject experiences increased resistance to stress-induced pathology.

7. The method according to any one of the preceding claims, wherein the non-invasive brain stimulation comprises audiovisual flicker.

8. The method according any one of the preceding claims, whereby the exposure of the subject to non-invasive brain stimulation is for the prophylactic treatment of stress.

9. The method according to any one of the preceding claims, wherein the non-invasive brain stimulation occurs at a frequency of about 20 Hz.

10. The method according to any one of the preceding claims, comprising exposing the subject to the non-invasive brain stimulation for about 15 to 90 minutes per day for seven or more days.

11. The method according to any one of the preceding claims, wherein the exposure of the subject to the non-invasive brain stimulation occurs at a fixed time on a daily basis.

12. The method according to any one of the preceding claims further comprising assessing the efficacy of the non-invasive brain stimulation in the subject following the exposure of the subject to the sensory stimulation.

13. The method according to claim 12, wherein the assessment includes performing a stress assessment of the subject.

14. The method according to claim 12 or claim 13, wherein the assessment includes a measurement of one or more biomarkers of stress-induced pathology in the subject.

15. The method according to claim 14, wherein the biomarkers include cytokines.

16. The method according to any one of claims 12-14, further comprising, based on the results of the assessment, determining whether to modulate one or more parameters of the sensory stimulation.

17. The method according to claim 16, wherein the parameters include frequency of the non-invasive brain stimulation, duration of the non-invasive brain stimulation per treatment episode, time of day of the non-invasive brain stimulation, or any combination thereof.

18. The method according to claim 16 or claim 17, further comprising exposing the subject to sensory brain in which the one or more of the parameters of the non-invasive brain stimulation have been modulated.

19. The method according to any one of the preceding claims, wherein the non-invasive brain stimulation reduces risk of or expression of anxiety, depression, aggression, anhedonia, decreased cognitive performance due to stress, or neurodegenerative disease in the subject.

20. The method according to any one of the preceding claims, wherein the stress-induced pathology is synaptic loss or neuronal atrophy.

21. The method according to any one of the preceding claims, wherein the non-invasive brain stimulation increases synaptic marker expression.

22. The method according to any one of the preceding claims wherein the non-invasive brain stimulation alters microglia.

23. The method according to any one of the preceding claims, wherein the non-invasive brain stimulation alters cytokine expression.

24. The method according to any one of the preceding claims, wherein the stress-induced pathology is immune dysregulation.

25. The method according to any one of the preceding claims, wherein the subject is not currently subject to a diagnosis of a neurodegenerative disease.

26. The method according to any one of the preceding claims, wherein the subject was selected for treatment by exposure to the brain stimulation by assessing the subject's susceptibility to stress-induced pathology.

27. The method according to claim 26, wherein the assessment of the subject's susceptibility to stress-induced pathology includes an anxiety assessment, an anhedonia assessment, a measurement of one or more biomarkers of stress pathology in the subject, or a combination thereof.

28. The method according to any one of the preceding claims, wherein the frequency of the brain stimulation is selected based on the stress-induced pathology to which the subject is susceptible.

29. The method according to any one of the preceding claims, wherein the frequency of the brain stimulation is selected based on the identity of a region within the subject's brain for which brain stimulation is desired.

30. The method according to claim 29, wherein the frequency of the brain stimulation is selected for stimulation of the subject's hippocampus (HPC), amygdala (AMY), prefrontal cortex (PFC), nucleus accumbens (NAc), or any combination thereof.

31. The method according to any one of the preceding claims, wherein the subject is in remission from a neuropsychiatric disorder.

32. The method of any one of the preceding claims, wherein the selected frequency for a female subject is greater than the selected frequency for a male subject.

33. The method of any one of the preceding claims, wherein the selected frequency is in the range of about 10 Hz to about 20 Hz for a male subject.

34. The method of claim 33, wherein the selected frequency is 10 Hz.

35. The method of any one of claims 1-32, wherein the selected frequency is in the range of about 30 to about 40 Hz for a female subject.

36. The method of claim 35, wherein the selected frequency is 40 Hz.

37. The method of any one of the preceding claims, wherein the subject is not currently subject to a diagnosis of a neurological or neuropsychiatric disorder.

38. The method of any one of the preceding claims, wherein the subject is selected for exposure to the brain stimulation based on (i) the subject's previous exposure to a stressor, (ii) the subject's contemporaneous exposure to a stressor, or (iii) an anticipated exposure of the subject to a future stressor

39. A method for increasing resilience to stress-induced pathology in a subject comprising:
exposing the subject to non-invasive brain stimulation that is effective to drive oscillations, rhythmic electrical activity, or both in the subject at a target frequency of about 5-100 Hz, wherein the target frequency is based on the subject's gender,
wherein the subject is not currently subject to a diagnosis of a neurological or neuropsychiatric disorder, and wherein the subject has been assessed as having an elevated genetic risk for a neurological or neuropsychiatric disorder.

40. A method for treating a neurological or neuropsychiatric disorder in a subject for whom an antidepressant or anti-anxiety medication is contraindicated or who is resistant to treatment with an antidepressant or anti-anxiety medication comprising exposing the subject to brain stimulation that is effective to drive oscillations, rhythmic electrical activity, or both in the subject at a target frequency of about 5-100 Hz, wherein a target frequency is based on the subject's gender.

41. The method according to claim 35, wherein the subject is pregnant or nursing.

42. A method for treating dysregulation of classic complement signaling, dysregulation of microglia activity, neuroinflammation, or pathological synaptic change in a subject comprising:
exposing the subject to brain stimulation that is effective to drive oscillations, rhythmic electrical activity, or both in the subject at a target frequency of about 5-100 Hz, wherein a target frequency is based on the subject's gender,
wherein the subject is selected for exposure to the brain stimulation based on a determination of neuroinflammation, dysregulation of classic complement signaling, dysregulation of microglia activity, or pathological synaptic change in the subject, or,
wherein the subject is selected for exposure to the brain stimulation based on a determination that the subject has an elevated genetic risk for a condition having a classic complement component.

43. The method according to claim 37, wherein the dysregulation of classic complement signaling is characterized by elevated classic complement signaling relative to a reference value corresponding to a normal level of classic complement signaling.

44. The method according to claim 37, wherein the dysregulation of classic complement signaling is characterized by decreased classic complement signaling relative to a reference value corresponding to a normal level of classic complement signaling.

45. The method according to claim 37, wherein the dysregulation of microglia activity is characterized by elevated microglia activity relative to a reference value corresponding to a normal level of microglia activity.

46. The method according to claim 37, wherein the dysregulation of microglia activity is characterized by decreased microglia activity relative to a reference value corresponding to a normal level of microglia activity.

47. The method according to claim 37, wherein the subject is selected for exposure to the brain stimulation based on a determination that the subject has an elevated genetic risk for bipolar disorder, amyotrophic lateral sclerosis, Parkinson's disease, attention deficit-hyperactivity disorder, obsessive-compulsive disorder, multiple sclerosis, systemic lupus, autism, inflammatory bowel disease, type-2 diabetes, or age-related macular degeneration.

48. A method for increasing resilience to stress-induced pathology in a subject comprising:
exposing the subject to brain stimulation that is effective to drive oscillations, rhythmic electrical activity, or both in the subject at a frequency of about 5-100 Hz,
wherein the subject is not currently subject to a diagnosis of a neurological or neuropsychiatric disorder, and,
wherein the subject is selected for exposure to the brain stimulation based on (i) the subject's previous exposure to a stressor, (ii) the subject's contemporaneous exposure to a stressor, or (iii) an anticipated exposure of the subject to a future stressor.

49. The method of any one of the preceding claims, wherein the method treats neuropsychiatric disease, neuropsychiatric symptoms, neurological disease, or neurological symptoms in the subject.

50. The method of claim 49, wherein the treatment is prophylactic.
